SEQUENCE LISTING (45 sequences)

SEQ ID NO: 1 - protein, 398 aa, Saccharomyces cerevisiae
MALEGLRKKYKTRQELVKALTPKRRSIHLNSNGHSNGTPCSNADVLAHIKHFLSLAANSLEQHQQPISIVFQNKKKKGDTSSPDIHTTLDFPLNGPHLCTHQFKLKRCAILLNLLKVVMEKLPLGKNTTVRDIFYSNVELFQRQANVVQWLDVIRFNFKLSPRKSLNIIPAQKGLVYSPFPIDIYDNILTCENEPKMQKQTIFPGKPCLIPFFQDDAVIKLGTTSMCNIVIVEKEAVFTKLVNNYHKLSTNTMLITGKGFPDFLTRLFLKKLEQYCSKLISDCSIFTDADPYGISIALNYTHSNERNAYICTMANYKGIRITQVLAQNNEVHNKSIQLLSLNQRDYSLAKNLIASLTANSWDIATSPLKNVIIECQREIFFQKKAEMNEIDARIFEYK

SEQ ID NO: 2 - protein, 398 aa, artificial
  Spo11-Y135F Saccharomyces cerevisiae
MALEGLRKKYKTRQELVKALTPKRRSIHLNSNGHSNGTPCSNADVLAHIKHFLSLAANSLEQHQQPISIVFQNKKKKGDTSSPDIHTTLDFPLNGPHLCTHQFKLKRCAILLNLLKVVMEKLPLGKNTTVRDIFFSNVELFQRQANVVQWLDVIRFNFKLSPRKSLNIIPAQKGLVYSPFPIDIYDNILTCENEPKMQKQTIFPGKPCLIPFFQDDAVIKLGTTSMCNIVIVEKEAVFTKLVNNYHKLSTNTMLITGKGFPDFLTRLFLKKLEQYCSKLISDCSIFTDADPYGISIALNYTHSNERNAYICTMANYKGIRITQVLAQNNEVHNKSIQLLSLNQRDYSLAKNLIASLTANSWDIATSPLKNVIIECQREIFFQKKAEMNEIDARIFEYK

SEQ ID NO: 3 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - protein, 1229 aa, Unknown
  Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHK

SEQ ID NO: 5 - protein, 1300 aa, artificial
  dead FnCpf1
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 6 - protein, 1229 aa, artificial
  dead LbCpf1
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHK

SEQ ID NO: 7 - protein, 7 aa, artificial
  signal de localisation nucl?aire (SLN)
PKKKRKV

SEQ ID NO: 8 - protein, 20 aa, artificial
  Cell penetrating peptide sequence derived from the TAT protein of HIV-1
GRKKRRQRRRPPQPKKKRKV

SEQ ID NO: 9 - protein, 19 aa, artificial
  Peptide sequence derived from the TLM sequence of the human hepatitis B virus
PLSSIFSRIGDPPKKKRKV

SEQ ID NO: 10 - protein, 362 aa, Arabidopsis thaliana
MEGKFAISESTNLLQRIKDFTQSVVVDLAEGRSPKISINQFRNYCMNPEADCLCSSDKPKGQEIFTLKKEPQTYRIDMLLRVLLIVQQLLQENRHASKRDIYYMHPSAFKAQSIVDRAIGDICILFQCSRYNLNVVSVGNGLVMGWLKFREAGRKFDCLNSLNTAYPVPVLVEEVEDIVSLAEYILVVEKETVFQRLANDMFCKTNRCIVITGRGYPDVSTRRFLRLLMEKLHLPVHCLVDCDPYGFEILATYRFGSMQMAYDIESLRAPDMKWLGAFPSDSEVYSVPKQCLLPLTEEDKKRTEAMLLRCYLKREMPQWRLELETMLKRGVKFEIEALSVHSLSFLSEVYIPSKIRREVSSP

SEQ ID NO: 11 - protein, 487 aa, Oryza sativa japonica
MDDSTDDDSYHPRKHYAYDRQVSSSRWRTSREYIRGPGPETHTTESAQDGQDPPAGVYSYGYFSGSGNDPQVQGHFVPEIQKYNPYVIFKGEQLPVPIWELPEEKVQDFHDRYFIAKDKSRVEARKTLNRLLEGNINTIERGHGYKFNIPKYTDNMEFNEEVKVSLAKAGKTISRSFCNANQREVASRTGYTIDLIERTLGAGLNISKRTVLYTNKDLFGDQSKSDQAINDICALTNIRRGSLGIIAAEKGIVVGNIFLELTNGKSISCSIGVQIPHRLDQIKDVCVEIGSRNIEYILVVEKHTMLNYLLEMDYHTNNNCIILTGCGMPTLQTRDFLRFLKQRTGLPVFGLCDPDPEGISILATYARGSCNSAYDNFNISVPSICWVGLSSSDMIKLNLSETNYSRLSREDKTMLKNLWQDDLSDVWKRRIEEMISFDKKASFEAIHSLGFDYFATNLLPDMINKVREGYVQVQEKKEPQDTEASED

SEQ ID NO: 12 - protein, 381 aa, Oryza sativa japonica
MAGREKRRRVAALDGEERRRRQEEAATLLHRIRGLVRWVVAEVAAGRSPTVALHRYQNYCSSASAAAASPCACSYDVPVGTDVLSLLHRGSHASRLNVLLRVLLVVQQLLQQNKHCSKRDIYYMYPSIFQEQAVVDRAINDICVLFKCSRHNLNVVPVAKGLVMGWIRFLEGEKEVYCVTNVNAAFSIPVSIEAIKDVVSVADYILIVEKETVFQRLANDKFCERNRCIVITGRGYPDIPTRRFLRYLVEQLHLPVYCLVDADPYGFDILATYKFGSLQLAYDANFLRVPDIRWLGVFTSDFEDYRLPDCCLLHLSSEDRRKAEGILSRCYLHREAPQWRLELEAMLQKGVKFEIEALSACSISFLSEEYIPKKIKQGRHI

SEQ ID NO: 13 - protein, 381 aa, Oryza sativa indica; identical to SEQ ID NO: 12

SEQ ID NO: 14 - protein, 442 aa, Brassica campestris
MSEKKRRGGAGAGAASGSASKKPRVSTAASYAESLRSKLRPDASILATLRSLASACSKSKPAGSSSSSSSASKALAAEDDPAASYIVVADQDSASVTSRINRLVLAAARSILSGRGFSFAVPSRAASNQVYLPDLDRIVLVRRESARPFANVATARKATITARVLSLVHAVLRRGIHVTKRDLFYTDVKLFGDQAQSDAVLDDVSCMLGCTRSSLHVVASEKGVVVGRLTFADDGDRIDCTRMGVGGKAIPPNIDRVSGIESDALFILLVEKDAAFMRLAEDRFYNRFPCIILTAKGQPDVATRLFLRRLKVELKLPVLALVDSDPYGLKILSVYMCGSKNMSYDSANLTTPDIKWLGVRPSDLDKYRVPEQCRLPMTDHDIKVGKELLEEDFVKQNEGWVKELETMLRTRQKAEIQALSSFGFQYLTEVYLPLKLQQQDWI

SEQ ID NO: 15 - protein, 362 aa, Brassica campestris
MEGNFQISETTNLLRKIKEFTRSIVEDLAEGKSPEISINRFRNYCNDPEADCFCSSDEPKGREILTLRKRSQTYRIDMLLRVLLIVQQLLQENRHGSKRDIYYMHPSAFKAQSVVDRAIADICILFQCSRYNLNVVSVGNGLVMGWLKFREAGRKFDCLSSLNTAFPVPVLVEEVEDIVSLAEYILVVEKETVFQRLANDMFCKTNRCIVVTGRGYPDVSTRRFLRLLMEKLQLPVHCLVDCDPYGFEILATYRFGSMQMAYDIESLRAPEMKWLGAFPSDSDIYGVPHQCLLPLTEEDKKRTEAMLLRCYLKREMPQWRLELETMLKRGVKFEIEALSVHSLSFLSEVYIPSKIRSEGSFH

SEQ ID NO: 16 - protein, 380 aa, Brassica campestris
MEEDASMKFFSDQNTSYADILPPREVRARIEVSVLNLLRVLNSPDPAISDLSLINRKRSNSCINQGILTDVSYIFLSTSFTKSSLTNAKTAKAFVRVWKVMEMCFQILLQEKRVTQRELFYKLLCDSPDLFSSQIEVNRSVQDVVALLRCSRFSLGIMASTRGLVAGRLYLQEPGKEPVDCSACGASGFPISGDLNLLDNTVMTSDARYIILVEKHAIFHRLVEDRVFNHIPCVFITAKGYPDIATRFFLHRMSITFPHLPILALVDWNPAGLAILCTFKFGSIGMGLEAYRYACNVKWIGLRGDDLNLIPEESLVPLKAKDSQIAKSLLSSKILQENYREELSLMIETGKRAEIEALYCHGYSYLGKYIATKIVQGKYI

SEQ ID NO: 17 - protein, 382 aa, Zea mays
MAEANVAAASLFGADGRLCSADILAPPEVRARIEVAVLNFLAALASPSSPAISVLPLISRSSANCSLRSGLLNDVSSVYLSYTFCKRSLTHNPKAFVRVWKVMEMCYKILGEGKLVQQRELFYKLLSDSPKYFSCQRHVNQAIQDVVSLLRCTRQSLGVMASSRGALIGRLVLHEPDGEQIDCSILGASGHAITGDLNLLSKLNLSSGSRYIIVVEKDAVFQRLAEDRLYNQLPCILITAKGYPDIATRFILHRLSQTFPNMPIFALVDWNPAGLAILCTYKYGSISMGLESYRYACNVKWLGVRGGDLHLIPEDAFQELKPRDLQIAKSLMSSKFLQESHRAELALMVERGKRADIEALYSHGFDFLGKYIARKIVQGDYI

SEQ ID NO: 18 - protein, 383 aa, Zea mays
MAGRDKRRRAAPLEGDEQQLRRRLEEAALLLRRIKGLVRWIVEEVAAGRSPSIVLHRYRNYCSSADSASPSPCACSYDIPVGTDVLSLLHKDYHTSRLNVLLRVLFVVQHLLQQNKHCSKRDIYYMYPSIFVEVAVVDRAINDICILFKCSRHNLNVVPVVKGLVMGWIRFMEGEKKVYCITSVNAAFSIPVDIEAIKDVVSVAHYILVVEKETVFQRLANDKFCERNRCIVITGRGYPDIPTRRFLRYLVELLHLPAYCLVDSDPYGFDILATYKFGSLQLAHDANLLRVPDIRWLGVFTSDFEEYCLPDCCLLRLSPEDRRKAEGILARCYLHREAPEWRSELEAMLQKGVKFEIEALSANSISFLSHEYIPQKIKQGMHL

SEQ ID NO: 19 - protein, 218 aa, Capsicum baccatum
MGWLRYAESGRKIDCISNPNTAYPIPVHVEEVNDILSVAQYVLVVEKESVFQRLANDQFCKRNRCVVITGRGYPDVPTRRFLRLLIDKLHLPVYCLVDCDPYGFDILTTYKFGSLQMAYDATFLQVSEIQWLGVFVQDSDNYSIPQQCLLPLTVEDKRKVKAMLHRCYLQREVPKWRFELELLLYKGVKFEIEALSVHSLTFLSHEYLPSKINNGGYI

SEQ ID NO: 20 - protein, 209 aa, Capsicum baccatum
MRNASEGQEGFTRSIVEDLARGNAPLIYIDRFRNYCTGTSGNCSCSSGLPTGKEAISLKRECHVRRLDILLRVLLIVQQLLQENRHGSKRDIYYMHPTVFKEQSVVDRAINDICILLQCSRHNLNVLHVLFTVKDSKILHIHAFDMGNGSSQVSERPREPHKFLALGGEFCLIFLLHDLSFSIAWGVRCLLEMGAYLYTVHISLQECAS

SEQ ID NO: 21 - protein, 382 aa, Carica papaya
MEDLGRSSMRFFSDQRICYADILPPNEARARIEVAVLNFLRILNSRDPAISSLPLVNRKWSNSRVSKGLLTDVSRIFLCRSFCIRSLMRVKATKAFVRVWKVMEMCFRILIQEKRVTQRELFYLLICDSPEYFSSQLEVNRTIQDVVALLQCSRYSLGIMASSRGIVAGRLLLQEPNQEVVDCSACGSSGYTISGDLNLIQTLILKTDARYIIVVEKHAIFQRLLEDGISNHIPSIIITAKGYPDIATRFLLHRMSSKFPGMPILALVDWNPAGLAILCTFKYGSIGMGLEAYRYACNVKWVGLRRDDLHLVPEQALIPLKPRDLQIAKSLMSSEVLQDEYREELASMIESGQRAEIEALFFHGYDYLGKYIAKKVVQANYI

SEQ ID NO: 22 - protein, 1352 aa, Unknown
  Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 23 - protein, 1477 aa, Unknown
  Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 24 - protein, 1307 aa, Unknown
  Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 25 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 26 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 27 - protein, 1300 aa, Francisella tularensis; identical to SEQ ID NO: 3

SEQ ID NO: 28 - protein, 1307 aa, Unknown; identical to SEQ ID NO: 24
  Acidaminococcus sp.

SEQ ID NO: 29 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 30 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 31 - protein, 1263 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 32 - protein, 1206 aa, Unknown
  Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 33 - protein, 1300 aa, Francisella tularensis; identical to SEQ ID NO: 3

SEQ ID NO: 34 - protein, 15 aa, artificial
  SLN
GGMAAPKKKRKVDGG

SEQ ID NO: 35 - protein, 8 aa, artificial
  linker1
GIHGVPAA

SEQ ID NO: 36 - protein, 12 aa, artificial
  linker2
PEFMAMEAPGIR

SEQ ID NO: 37 - protein, 31 aa, artificial
  6xHis-3xFlag Tag
HHHHHHGDYKDDDDKDYKDDDDKDYKDDDDK

SEQ ID NO: 38 - protein, 14 aa, artificial
  Tag V5 14 amino acids
GKPIPNPLLGLDST

SEQ ID NO: 39 - protein, 9 aa, artificial
  Tag V5 9 amino acids
IPNPLLGLD

SEQ ID NO: 40 - protein, 385 aa, Oryza sativa
MAEAGVAAASLFGADRRLCSADILPPAEVRARIEVAVLNFLAALTDPAAPAISALPLISRGAANRGLRRALLRDDVSSVYLSYASCKRSLTRANDAKAFVRVWKVMEMCYKILGEGKLVTLRELFYTLLSESPTYFTCQRHVNQTVQDVVSLLRCTRQSLGIMASSRGALIGRLVVQGPEEEHVDCSILGPSGHAITGDLNVLSKLIFSSDARYIIVVEKDAIFQRLAEDRIYSHLPCILITAKGYPDLATRFILHRLSQTYPNMPIFALVDWNPAGLAILCTYKYGSISMGLESYRYACNVKWLGLRGDDLQLIPQSAYQELKPRDLQIAKSLLSSKFLQDKHRAELTLMLETGKRAEIEALYSHGFDFLGKYVARKIVQGDYI

SEQ ID NO: 41 - protein, 383 aa, Zea mays
MAGRDKRRRAAPLEGDEQQLRRRLEEAALLLRRIKGLVRWIVEEVAAGRSPSIVLHRYRNYCSSADSASPSPCACSYDIPVGTDVLSLLHKDYHTSRLNVLLRVLFVVQQLLQQNKHCSKRDIYYMYPSIFVEVAVVDRAINDICILFKCSRHNLNVVPVVKGLVMGWIRFMEGEKKVYCITSVNAAFSIPVDIEAIKDVVSVAHYILVVEKETVFQRLANDKFCERNRCIVITGRGYPDIPTRRFLRYLVELLHLPAYCLVDSDPYGFDILATYKFGSLQLAHDANLLRVPDIRWLGVFTSDFEEYCLPDCCLLRLSPEDRRKAEGILARCYLHREAPEWRSELEAMLQKGVKFEIEALSANSISFLSDEYIPQKIKQGMHL

SEQ ID NO: 42 - protein, 382 aa, Zea mays
MAEANVAAASLFGADRRLCSADILAPPEVRGRIEVAVLNFLAALASPSSPAISVLPLISRSSANCSLRSGLLNDVSSVYLSYTFCKRSLTHNPKAFVRVWKVMEMCYKILGEGKLAQQRELFYKLLSDSPKYFSCQRHVNQAIQDVVSLLRCTRQSLGVMASSRGALIGRLVLHEPDGEQIDCSILGASGHAITGDLNLLSKLNLSSGSRYIIVVEKDAVFQRLAEDRLYNQLPCILITAKGYPDIATRFILHRLSQTFPNMPIFALVDWNPAGLAILCTYKYGSISMGLESYRYACNVKWLGVRGGDLHLIPEGAFQELKPRDLQIAKSLMSSKFLQESHRAELALMVDRGKRADIEALYSHGFDFLGKYIARKIVQGDYI

SEQ ID NO: 43 - DNA, 25 bp, artificial
  wild type target sequence pGAL2B,C
acggcgcagatatctccgcacggac

SEQ ID NO: 44 - DNA, 25 bp, artificial
  mutated target sequence pGAL2b,c
attccgcagatatctccgcatatac

SEQ ID NO: 45 - DNA, 25 bp, Artificial Sequence
  guide sequence targeting the Gal4UAS-B,C site
gtccgtgcggagatatctgcgccgt

(19) United States
(12) Patent Application Publication
(10) Pub. No.: US 2024/0263157 A1
(43) Pub. Date: Aug. 8, 2024

(54) USE OF A FUSION PROTEIN FOR INDUCING GENETIC MODIFICATIONS BY TARGETED MEIOTIC RECOMBINATION

(71) Applicants: MEIOGENIX, PARIS (FR); INSTITUT CURIE, PARIS CEDEX 05 (FR); CENTRE NATIONAL DE LA RECHERCHE SCIENTIFIQUE, PARIS (FR); SORBONNE UNIVERSITE, PARIS (FR)

(72) Inventors: ALEXANDRE SERERO, COLOMBES (FR); ALAIN NICOLAS, PARIS (FR)

(21) Appl. No.: 17/926,160

(22) PCT Filed: May 20, 2021
(86) PCT No.: PCT/FR2021/050917

§ 371 Date: Nov. 18, 2022

(30) Foreign Application Priority Data
May 20, 2020 (FR) 2005370

(51) Int. Cl. C12N 9/22; C12N 9/90; C12N 15/10; C12N 15/113; C12N 15/81

(52) U.S. Cl. CPC C12N 9/22; C12N 9/90; C12N 15/102; C12N 15/113; C12N 15/81; C07K 2319/21; C07K 2319/43; C12N 2310/20; C12Y 599/01

(57) ABSTRACT

The present invention relates to a fusion protein comprising a nuclease domain from the class 2 CRISPR system, in particular Cpf1, and a Spo11 domain, as well as the use of this protein in order to induce targeted meiotic recombinations in an eukaryotic cell.

### CROSS-REFERENCE TO RELATED APPLICATION

This application is the U.S. national stage application of International Patent Application No. PCT/FR2021/050917, filed May 20, 2021.

### REFERENCE TO SEQUENCE LISTING

The Sequence Listing for this application is labeled “Seq-List-replace-2.txt” which was created on May 11, 2023 and is 238,416 bytes. The entire content of the sequence listing is incorporated herein by reference in its entirety.

### TECHNICAL FIELD

The present invention pertains to the field of targeted genetic modifications in eukaryotes. It relates in particular to a process for improving or modifying a eukaryotic cell by inducing targeted meiotic recombinations.

### TECHNOLOGICAL BACKGROUND OF THE INVENTION

The modification of the genetic material of eukaryotic organisms has developed greatly in the past twenty years, and has found application in the field of plants, human and animal cells as well as microorganisms such as yeast for applications in the fields of agriculture, human health, agri-food and environmental protection.

Yeasts find their application in a wide variety of industrial fields. Because many species are harmless, yeasts are in particular used in the food industry as fermentation agent in baking, brewing, wine-making or distilling, or in the form of extracts as nutritional elements or flavoring agents. They can also find use in the industrial production of bioethanol or molecules of interest such as vitamins, antibiotics, vaccines, enzymes, or steroid hormones, or in processes for degrading cellulosic materials. Similarly, plants are used in many industrial fields, whether in the agri-food, cosmetic or pharmaceutical industries.

The diversity of industrial applications of yeast and plants implies that there is a constant demand for yeast strains and plant varieties with improved traits or, at least, adapted to a new use or new culture conditions. In order to obtain a eukaryotic cell or organism with a particular trait, the person skilled in the art can use sexual reproduction and select a hybrid cell or organism providing the desired combination of parental traits. This method is however random and the selection step can lead to significant delays, in particular in the case of yeasts and plants.

Alternatively, the skilled person can also modify the genetic heritage of a cell or an organism by a recombinant DNA technique. This modification can nevertheless constitute an impediment to its exploitation, whether for regulatory, sanitary or environmental reasons, in particular in the case of plants considered as genetically modified organisms (GMO).

A third alternative consists in causing a reassortment of alleles of paternal and maternal origin in the genome, during meiotic recombination. Meiotic recombination is an exchange of DNA between homologous chromosomes during meiosis. After DNA replication, recombination is initiated by the formation of double-strand breaks in one (or the other) of the chromatids of the homologous chromosomes, followed by the repair of these breaks, using a chromatid of the homologous chromosome as a template. However, meiotic recombinations have the disadvantage of being non-uniform. Indeed, the double-strand break sites at the origin of these recombinations are not distributed homogeneously in the genome. A distinction can thus be made between so-called ‘hot’ chromosomal regions where the frequency of recombination is high, and so-called ‘cold’ chromosomal regions where the frequency of recombination can be up to 100 times lower.

Spo11 is the protein that catalyzes double-strand breaks during meiosis. It acts in dimer form in cooperation with other partner proteins. At present, the factors determining the choice of double-strand break sites by Spo11 and its partners remain poorly understood.

The control of double-strand break formation and, hence, of meiotic recombinations, is crucial for the development of genetic engineering techniques. In particular, it has been shown that it is possible to modify the double-strand break formation sites by fusing Spo11 with the DNA-binding domain of the transcriptional activator Gal4 (Pecina et al, 2002 Cell, 111, pp 173-184). The Gal4BD-Spo11 and Gal4-Spo11 fusion proteins allow the introduction of double-strand breaks in so-called ‘cold’ chromosomal regions, at the level of Gal4 DNA binding sites. However, according to the Spo11-Gal4 system, the introduction of targeted double-strand breaks is conditioned by the presence of Gal4 binding sites, making it impossible to induce targeted meiotic recombination phenomena independently of specific binding sites.

Local stimulation of meiotic recombination at a number of chromosomal sites has been reported following binding of Spo11 to the deadCas9 protein (Sarno et al, Nucleic Acids Research, 45 pp e164). However, the Cas9 endonuclease and its guide RNA requires a specific guanine-rich sequence called PAM (5′-NGG-3′), which limits the potential uses of this system.

Thus, there remains a need to provide methods that allow easy and specific induction of meiotic recombination of genomic regions inaccessible to prior art techniques and that can be applied to a wide range of eukaryotic cells.

### SUMMARY OF THE INVENTION

The objective of the present invention is to propose a fusion protein as well as a process for inducing targeted meiotic recombinations in eukaryotic cells, preferably yeast or plant cells, in any region of the genome, preferably several different regions of the genome, independently of any known binding site, and in particular in so-called ‘cold’ chromosomal regions.

Thus, according to a first aspect, the present invention relates to a fusion protein comprising (i) a nuclease associated with a CRISPR system, preferably a class 2 CRISPR system, and (ii) a Spo11 protein or one of its partners involved in the formation and repair of double-strand breaks during meiosis, wherein the nuclease associated with the CRISPR system is not a Cas9 nuclease.

Preferably, the nuclease associated with a CRISPR system is not a class II and type II nuclease.

Preferably, the nuclease (i) is associated with a class II CRISPR system, preferably type V. Most preferably, the nuclease associated with a CRISPR system is a Cpf1 nuclease. In particular, said Cpf1 nuclease may be a Cpf1 nuclease comprising a sequence selected from the sequences SEQ ID NO: 3, 4 and 22 to 33, and the variants of said sequences having at least 80, 85, 90, 95, 96, 97, 98, or at least 99% identity with one of these sequences and a Cpf1 activity.

In certain embodiments, the fusion protein may comprise a nuclease associated with a CRISPR system which is deficient in nuclease activity. In particular, this nuclease may be a variant of a wild-type Cpf1 protein having at least 80, 85, 90, 95, 96, 97, 98, or at least 99% identity with said Cpf1 protein and in which the residue corresponding to the aspartate at position 832 of SEQ ID NO: 4 is substituted, preferably by an alanine. More particularly, this nuclease may be a variant of a Cpf1 protein selected from the sequences SEQ ID NO: 3, 4 and 22 to 33, having at least 80, 85, 90, 95, 96, 97, 98, or at least 99% identity with said sequence and in which the residue corresponding to the aspartate at position 832 of SEQ ID NO: 4 is substituted, preferably by an alanine.

In preferred embodiments, the fusion protein comprises a Spo11 protein. The Spo11 protein may especially be selected from the sequences of SEQ ID NO: 1, 10 to 21 and 40-42, and the variants thereof comprising a sequence having at least 80, 85, 90, 95, 96, 97, 98, or at least 99% identity with one of these sequences and a Spo11 activity.

In particular, the fusion protein may comprise a Spo11 protein deficient in nuclease activity. The Spo11 protein deficient in nuclease activity may be a variant of a wild-type Spo1l protein having at least 80%, 85%, 90%, 95%, 96%, 97%, 98%, or at least 99% identity with said Spo11 protein and in which the residue corresponding to the tyrosine at position 135 of SEQ ID NO: 1 is substituted, preferably by a phenylalanine. In particular, the Spo11 protein deficient in nuclease activity may be a variant of a Spo11 protein of one of the sequences SEQ ID NO: 1, 10 to 21 and 40-42, comprising a sequence having at least 80%, 85%, 90%, 95%, 96%, 97%, 98%, or at least 99% identity with one of these sequences and in which the residue corresponding to the tyrosine at position 135 of SEQ ID NO: 1 is substituted, preferably by a phenylalanine.

Alternatively, the fusion protein may comprise a partner of Spo11, preferably selected from the group consisting of Rec102, MTOPVIB/TOPOVIBL, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1, and Spp1, and orthologues thereof.

According to a second aspect, the present invention relates to a nucleic acid encoding the fusion protein defined above.

According to a third aspect, the present invention also relates to an expression cassette or vector comprising a nucleic acid as defined above.

According to a fourth aspect, the present invention also relates to a host cell, preferably non-human, comprising a fusion protein, a nucleic acid, a cassette or a vector as defined above.

Preferably, the host cell is a eukaryotic cell, even more preferably a yeast, plant, fungus or animal cell, and most preferably, the host cell is a plant cell or a yeast cell. In particular, the host cell is a plant cell, the plant preferably being of agronomic, horticultural, pharmaceutical or cosmetic interest, in particular vegetables, fruits, herbs, flowers, trees and shrubs.

The plant cell is preferably selected from monocotyledonous plants and dicotyledonous plants, more particularly preferably selected from the group consisting of rice, wheat, soybean, maize, tomato, onion, cucumber, lettuce, asparagus, carrot, turnip, Arabidopsis thaliana, barley, rapeseed, cotton, grapevine, sugarcane, beet, cotton, sunflower, oil palm, coffee, tea, cocoa, chicory, bell pepper, chili, lemon, orange, nectarine, mango, apple, banana, peach, apricot, sweet potato, yam, almond, hazelnut, strawberry, melon, watermelon, olive tree, potato, zucchini, eggplant, avocado, cabbage, plum, cherry, pineapple, spinach, apple, mandarin, grapefruit, pear, grape, clove, cashew nut, coconut, sesame, rye, hemp, tobacco, berries such as raspberry or blackcurrant, peanut, castor, vanilla, poplar, eucalyptus, green foxtail, cassava, and horticultural plants such as, for example, roses, tulips, orchids and geraniums. In particular, the plant cell may be selected from the group consisting of rice, wheat, soybean, maize, tomato, onion, cucumber, lettuce, asparagus, carrot, turnip, Arabidopsis thaliana, barley, rapeseed, cotton, grapevine, sugarcane, beet, cotton, sunflower, palm olive tree, coffee, tea, cocoa, chicory, bell pepper, chili, lemon, orange, nectarine, mango, apple, banana, peach, apricot, sweet potato, yam, almond, hazelnut, strawberry, melon, watermelon, olive tree, and horticultural plants such as roses, tulips, orchids and geraniums. Preferably, the plant cell is selected from the group consisting of rice, wheat, soybean, maize, and tomato.

According to a fifth aspect, the invention relates to a process for inducing targeted meiotic recombinations in a eukaryotic cell, preferably non-human, comprising
introduction into said cell of:
a) a fusion protein, a nucleic acid, an expression cassette, or a vector as described above; and
b) one or more guide RNAs or one or more nucleic acids encoding said guide RNAs, said guide RNAs comprising a nuclease binding RNA structure associated with a CRISPR system of the fusion protein and a sequence complementary to the targeted chromosomal region; and
induction of entry into prophase I of meiosis of said cell.

The present invention further relates, in a sixth aspect, to a process for generating variants of a eukaryotic organism, preferably non-human, comprising:
introduction into a cell of said organism of:
a) a fusion protein, a nucleic acid, an expression cassette, or a vector as described above; and
b) one or more guide RNAs or one or more nucleic acids encoding said guide RNAs, said guide RNAs comprising a nuclease binding RNA structure associated with a CRISPR system of the fusion protein and a sequence complementary to the targeted chromosomal region; and
induction of entry into prophase I of meiosis of said cell;
obtaining cell(s) with the desired recombination(s) in the targeted chromosomal region(s); and
genesis of a variant of the organism from said recombinant cell.

In a seventh aspect, the present invention also relates to a process for identifying or locating genetic information encoding a trait of interest in a eukaryotic cell genome, preferably non-human, comprising:
introduction into the eukaryotic cell of:
a) a fusion protein, a nucleic acid, an expression cassette, or a vector as described above; and
b) one or more guide RNAs or one or more nucleic acids encoding said guide RNAs, said guide RNAs comprising a nuclease binding RNA structure associated with a CRISPR system of the fusion protein and a sequence complementary to the targeted chromosomal region; and
induction of entry into prophase I of meiosis of said cell;
obtaining cell(s) with the desired recombination(s) in the targeted chromosomal region(s); and
analysis of the genotypes and phenotypes of the recombinant cells in order to identify or locate the genetic information encoding the trait of interest.

Preferably, the trait of interest is a quantitative trait of interest (QTL).

In an eighth aspect, the present invention finally relates to the use of a fusion protein, a nucleic acid, an expression cassette or a vector to (i) induce targeted meiotic recombinations in a eukaryotic cell, preferably non-human, (ii) generate variants of a eukaryotic organism, preferably non-human, and/or (iii) identify or locate genetic information encoding a trait of interest in a eukaryotic cell genome, preferably non-human.

Preferably, in the processes or uses according to the invention, the eukaryotic cell is a yeast plant or fungal cell or an animal cell, preferably a yeast, plant or fungal cell.

### BRIEF DESCRIPTION OF THE DRAWINGS

FIG. 1 illustrates the formation of meiotic double-strand breaks (DSBs) induced by the Cpf1-Spo11135F fusion protein and their repair by homologous recombination in the GAL2 gene promoter region.

FIG. 2 illustrates the ability of the Cpf1-Spo11^{Y135F} fusion protein to stimulate meiotic recombination in the target region of the GAL2 gene promoter.

### DETAILED DESCRIPTION OF THE INVENTION

The clustered regularly interspaced short palindromic repeats (CRISPR) system is a defense system demonstrated in bacteria and archaea against foreign DNA. These short fragments corresponding to the infectious agent are inserted into a series of CRISPR repeats and are used as CRISPR guide RNA (crRNA) to target the infectious agent in subsequent infections. This system is essentially based on the association of a CRISPR-associated (Cas) endonuclease protein and a ‘guide’ RNA (gRNA or sgRNA) responsible for the specificity of the cleavage site. It allows DNA double-strand breaks (DSBs) to be made at the sites targeted by the CRISPR system.

There are five main types of CRISPR systems that differ in the repertoires of CRISPR-associated genes, the organization of Cas operons and the structure of repeats within CRISPR arrays. These five types of systems have been divided into two classes: class I comprising types I, III and IV that use a multimeric crRNA effector module and class II comprising types II, V and VI that use a monomeric crRNA effector module.

Class II and type II CRISPR systems, predominantly represented by the Cas9 and Csn2 nucleases, comprise a small trans-acting crRNA (tracrRNA) that pairs with each pre-CRISPR RNA (crRNA) repeat to form a double-stranded RNA [tracrRNA:crRNA] cleaved by RNase III in the presence of the endonuclease.

Class II and type VI CRISPR systems are represented by the C13a (previously known as C2c2), C13b, and C13c proteins. The CRISPR-C13 system was discovered in the bacterium Leptotrichia shahii (Abudayyeh et al, Science 2016; 353(6299): aaf5573) and is analogous to the CRISPR-Cas9 system. However, unlike Cas9, which targets DNA, the C13 proteins target and cleave single-stranded RNA.

Class II and type V CRISPR-Cas systems are, among others, represented by the nuclease Cpf1 (also called Cas12a) recently discovered in the bacterium Francisella novicida (Zetsche B, Cell, 2015; 163: 759-771), and the nucleases C2c1 (also called Cas12b) and C2c3 identified in Alicyclobacillus acidoterrestris (Shmakov et al, Molecular Cell, 2015 Volume 60, Issue 3, P385-397). Cpf1 contains a mixed alpha/beta domain, a RuvC-I domain followed by a helical region, a RuvC-II domain and a zinc-finger domain. A functional CRISPR-Cpf1 system does not require a tracrRNA but only a crRNA. In particular, a 42-44 nucleotide crRNA with a direct repeat sequence of about 19 nucleotides followed by a 23-25 nucleotide protospacer sequence is sufficient to guide the Cpf1 endonuclease to the target nucleic acid.

The Cpf1-crRNA complex cleaves target DNA or RNA by identifying a 5′-YTN-3′ or 5′-TTTN-3′ PAM motif adjacent to the proto-spacer (where ‘Y’ is a pyrimidine and ‘N’ is any nucleobase), as opposed to the guanine-rich PAM motif (5′-NGG-3′) targeted by Cas9. Recognition of a thymidine-rich PAM extends the number of sites targeted by the CRISPR technique to A-T rich regions devoid of PAM motifs and allows its use in cells with a G-rich genome, thus limiting the occurrence of aspecific targeting or ‘off-targets’.

After identification of the PAM motif, Cpf1 introduces a double-strand break releasing sticky ends, typically generating 4 or 5 overhanging nucleotides. This type of break differs from the breaks generated by class II and type II nucleases such as Cas9, which generate clean ends after cleavage, and allows, in the manner of Velcro, directional insertions of genes, analogous to those made by conventional restriction enzymes. Cpf1 cleaves the DNA 18-23 bp downstream of the PAM site, resulting in no disruption of the recognition sequence after double-strand break (DSB) repair. Consequently, Cpf1 allows multiple DNA cleavage cycles and provides an increased possibility of achieving the desired genomic modification.

The inventors have demonstrated that it is possible to modify the CRISPR-Cpf1 system in order to induce targeted meiotic recombinations in a eukaryotic cell, and in particular in a yeast or a plant cell. They have indeed shown that the combined expression of a fusion protein comprising a Cpf1 domain and a Spo11 domain and a guide RNA allowed, surprisingly, the induction of targeted meiotic recombination through double-strand breaks during prophase I of meiosis and the repair of these breaks.

This system has never been used to target meiotic recombination sites in any organism.

Thus, according to a first aspect the present invention relates to a fusion protein comprising (i) a first domain (CRISPR domain) which is a nuclease associated with a CRISPR system, preferably a class II CRISPR system, and (ii) a second domain (Spo11 domain) which is a Spo11 protein or one of the Spo11 partners involved in the formation and repair of double-strand breaks during meiosis.

As used herein, the term ‘fusion protein’ refers to a chimeric protein comprising at least two domains derived from the combination of different proteins or protein fragments. The nucleic acid encoding this protein is obtained by juxtaposing the regions encoding the proteins or protein fragments in such a way that they are in phase and transcribed on the same mRNA. The different domains of the fusion protein can be directly adjacent or separated by linker sequences that introduce a certain structural flexibility into the construct.

The fusion protein according to the present invention comprises a first domain (CRISPR domain) which is a nuclease associated with a CRISPR system and a second domain (Spo11 domain) which is a Spo11 protein or one of the Spo11 partners involved in the formation and repair of double-strand breaks during meiosis.

Spo11 is a protein related to the catalytic subunit A of a type II topoisomerase found in archaea (Bergerat et al, Nature, vol. 386, pp 414-7). It catalyzes DNA double-strand breaks that initiate meiotic recombination. It is a highly evolutionarily conserved protein for which homologues exist in all eukaryotes. Spo11 is active as a dimer formed by two subunits, each of which cleaves a strand of DNA. Although essential, Spo11 does not act alone to generate double-strand breaks during meiosis. In the yeast S. cerevisiae, for example, it cooperates with the proteins, Rec102, MTOPVIB, Rec103/Sk18, Rec104, Rec114, Mer1, Mer2/Rec107, Mei4, Mre2/Nam8, Mre11, Rad50, Xrs2/Nbs1, Hop1, Red1, Mek1, Set1, and Spp1 as described in the papers by Keeney et al (2001 Curr. Top. Dev. Biol, 52, pp 1-53), Smith et al (Curr. Opin. Genet. Dev, 1998, 8, pp 200-211) and Acquaviva et al (2013 Science, 339, pp 215-218). It has been shown that targeting Spo11 to a given site is sufficient to trigger the meiotic recombination process (Pecina et al, 2002 Cell, 111, pp 173-184; Acquaviva et al, 2013 Science, 339, pp 215-218). It should be noted that multiple Spo11 protein homologues can co-exist in the same cell, in particular in plants.

The Spo11 protein, fragment or domain thereof as used in the present invention, can be obtained from any known Spo1l protein such as the Saccharomyces cerevisiae Spo11 protein (Gene ID: 856364, NCBI accession number: NP_011841 (SEQ ID NO: 1), Esposito and Esposito, Genetics, 1969, 61, pp 79-89), the Arabidopsis thaliana AtSpo11-1 and AtSpo11-2 protein (Grelon M. et al, 2001, Embo J., 20, pp 589-600), the murine mSpo11 protein (Baudat F et al, Molecular Cell, 2000, 6, pp 989-998), the C. elegans Spo11 protein or the Drosophila Spo11 protein meiW68 (McKim et al, 1998, Genes Dev, 12(18), pp 2932-2942). Of course, these examples are not limiting and any known Spo11 protein can be used in the fusion protein according to the invention. Preferably, the Spo11 protein is obtained from one of the Spo11 proteins of the eukaryotic cell of interest.

According to a preferred embodiment, the Spo11 domain comprises, or consists of, a Spo11 protein, preferably a wild-type Spo11 protein, in particular a Spo11 protein of the eukaryotic cell of interest, or a sequence having at least 80%, 85%, 90%, 95%, 96%, 97%, 98%, or at least 99% identity with said Spo11 protein and having Spo11 activity.

According to a particular embodiment, the Spo11 domain comprises, or consists of, a Spo11 protein, preferably a Saccharomyces cerevisiae Spo11 protein, such as, for example, the protein of sequence NP_011841 (SEQ ID NO: 1) or a sequence having at least 80, 85, 90, 95, 96, 97, 98, or at least 99% identity with a Spo11 protein, preferably with a Saccharomyces cerevisiae Spo11 protein, or a related protein carrying motifs conserved with the Spo11 protein, in particular the protein of sequence SEQ ID NO: 1.

According to a particular embodiment, multiple fusion proteins according to the invention comprising different Spo11 domains may be introduced into the same cell. In particular, when multiple Spo11 homologues exist in the eukaryotic cell of interest, the different fusion proteins may comprise different Spo11 homologues. By way of example, two fusion proteins according to the invention comprising Arabidopsis thaliana Spo11-1 and Spo11-2 domains, respectively, may be introduced into the same cell, preferably into the same Arabidopsis thaliana cell. Also by way of example, one or more fusion proteins according to the invention comprising rice Spo11-1, Spo11-2, Spo11-3 and/or Spo11-4 domains can be introduced into the same cell, preferably into the same rice cell. Numerous Spo11 homologues have been identified in different species, in particular in plant species (Sprink T and Hartung F, Frontiers in Plant Science, 2014, Vol. 5, Article 214, doi: 10.3389/fpls.2014.00214; Shingu Y et al, BMC Mol Biol, 2012, doi: 10.1186/1471-2199-13-1). The person skilled in the art can easily identify Spo11 homologues in a given species, in particular by means of well-known bioinformatics techniques.

According to a preferred embodiment, the Spo11 domain comprises, or consists of, a plant Spo11 protein, in particular selected from: Arabidopsis thaliana Spo11 proteins, for example as described under UniProt entry Q9M4A2-1 (SEQ ID NO: 10), Oryza sativa (rice) Spo11 proteins, for example as described by Fayos I. et al, 2019 Plant Biotechnol J. November; 17(11):2062-2077 and under UniProt entries Q2QM00 (SEQ ID NO: 11), Q7Y021 (SEQ ID NO: 12), Q5ZPV8 (SEQ ID NO: 13), A2XFC1 (SEQ ID NO: 14), and UniProt, Q6ZD95 (SEQ ID NO: 40), Brassica campestris (mustard) Spo11 proteins, for example as described under UniProt entries A0A024AGF2 (SEQ ID NO: 15) and A0A024AHI2 (SEQ ID NO: 16), Zea mays (maize) Spo11 proteins, for example as described under UniProt entries B6UAQ8 (SEQ ID NO: 17) and B6TWI5 (SEQ ID NO: 18), A0A1P8W169-1 (SEQ ID NO: 41) and A0A1P8W163 (SEQ ID NO: 42), Capsicum baccatum (pepper tree) Spo11 proteins, for example as described under entries A0A2G2WFG5 (SEQ ID NO: 19) and A0A2G2WFH4 (SEQ ID NO: 20), Carica papaya (papaya) Spo11 protein, for example as described under UniProt entry A0A024AG98 (SEQ ID NO: 21).

In particular, the Spo11 domain may comprise, or consist of, a Spo11 protein selected from any of the above-mentioned Spo11 proteins, preferably the sequences of SEQ ID NO: 1, 2, and 10 to 21 and 40-42, and variants thereof comprising a sequence having at least 80, 85, 90, 95, 96, 97, 98, or at least 99% identity with one of these sequences and Spo11 activity. More particularly, the Spo11 domain may comprise, or consist of, a Spo11 protein selected from the sequences of SEQ ID NO: 1, 10 to 21 and 40-42, and the variants thereof comprising a sequence having at least 80, 85, 90, 95, 96, 97, 98, or at least 99% identity with one of these sequences and a Spo11 activity. Preferably the Spo11 domain may comprise, or consist of, a Spo11 protein selected from the sequences of SEQ ID NO: 10 to 21 and 40-42, and the variants thereof comprising a sequence having at least 80%, 85%, 90%, 95%, 96%, 97%, 98%, or at least 99% identity with one of these sequences and a Spo11 activity.

As used herein, the term ‘Spo11 activity’ refers to the ability of a protein to induce double-strand breaks during prophase I of meiosis and/or the ability of a protein to recruit one or more Spo11 partners as defined below. Preferably, this term refers to the ability of a protein to recruit one or more Spo11 partners and, optionally, the ability of a protein to induce double-strand breaks during prophase I of meiosis. The ability of a protein to induce double-strand breaks during prophase I of meiosis and to recruit one or more partners of Spo11 may be readily tested by the person skilled in the art, for example by a complementation test in a yeast or in a plant in which the endogenous Spo11 protein has been deactivated. If the protein possesses this ability, this organism will produce viable spores. The ability of one protein to recruit another, for example of a protein to recruit a partner of Spo11 or of a partner of Spo11 to recruit a Spo11 protein, may readily be tested by the person skilled in the art, by means of conventional techniques, such as the double hydride technique or the ChTP (chromatin immunoprecipitation) technique. The ability of a protein to induce double-strand breaks during prophase I of meiosis may be readily tested by the person skilled in the art, by means of conventional techniques, for example by Southern blot or by sequencing of the oligonucleotides associated with a protein, in particular Spo11. For a wild-type Spo11 protein or a variant endowed with nuclease activity, the term “Spo11 activity” refers preferably to the ability of a protein to induce double-strand breaks during prophase I of meiosis and the ability of a protein to recruit, directly or indirectly, one or more partners of Spo11 as defined below. For a Spo11 variant which exhibits deficient nuclease activity, the term “Spo11 activity” refers preferably to the ability of a protein to recruit one or more partners of Spo11 as defined below.

According to a particular aspect, the Spo11 domain of the fusion protein comprises a variant of a Spo11 protein, preferably a variant whose nuclease activity has been abolished, reduced, or enhanced relative to the wild-type Spo1l protein.

In an embodiment, the Spo11 domain of the fusion protein according to the invention is endowed with nuclease activity and responsible for double-strand breaks. This domain may consist of a Spo11 protein or a fragment thereof capable of inducing DNA double-strand breaks.

Alternatively, the Spo11 domain may comprise a variant of a Spo11 protein that has deficient nuclease activity also referred to as ‘dead Spo11’ or ‘dSpo11’. Thus, the fusion protein used in the present invention may comprise a domain that is a nuclease associated with a CRISPR system and a Spo11 domain which is a Spo11 variant with deficient nuclease activity. In this embodiment, when several fusion proteins according to the invention are introduced into the eukaryotic cell, the different Spo11 domains are preferably all deficient in nuclease activity.

As used herein, the term ‘nuclease activity’ refers to the enzymatic activity of an endonuclease that has an active site for creating cuts within DNA or RNA chains, preferably DNA double-strand breaks. ‘Deficient nuclease activity’ means, particularly, a reduced, diminished or non-existent nuclease activity in particular relative to the nuclease activity of the wild-type protein from which the variant is derived.

According to a particular embodiment, the DNA or RNA cleavage capacity and/or the hydrolase activity of the nuclease is reduced or diminished, in particular relative to the nuclease activity of the wild-type protein. Preferably, the nuclease activity of a Spo11 domain with deficient nuclease activity is reduced by at least 50, 55, 60, 65, 70, 75, 80, 85, 90, 95 or 100%, relative to the nuclease activity of the wild-type Spo11 domain. Particularly preferably, a Spo11 domain with deficient nuclease activity is a Spo11 domain that is unable to generate double-strand breaks.

In particular, the Spo11 domain with deficient nuclease activity may comprise a mutated catalytic site which induces deficient nuclease activity, the mutation negatively impacting the nuclease or hydrolytic capacity of the Spo11 domain. Preferably, the Spo11 domain may comprise, or consist of, a mutant Spo11 protein in which the residue corresponding to tyrosine at position 135 of SEQ ID NO: 1 is substituted, preferably by a phenylalanine. A Spo11 protein with such a substitution is unable to induce DNA double-strand breaks (Bergerat et al, Nature, vol. 386, pp 414-417) and may in particular have a sequence as described in SEQ ID NO: 2. The residue corresponding to the tyrosine at position 135 of SEQ ID NO: 1 in the sequence of a Spo11 protein may readily be identified by conventional techniques of sequence alignment.

Accordingly, the Spo11 domain may be a variant of a Spo11 protein, preferably of a wild-type Spo11 protein, in particular of the eucaryotic cell of interest, having at least 80%, 85%, 90%, 95%, 96%, 97%, 98%, or at least 99% identity with said Spo11 protein and in which the residue corresponding to the tyrosine at position 135 of SEQ ID NO: 1 is substituted, preferably by a phenylalanine. In particular, the Spo11 domain can be a variant of the sequences SEQ ID NO: 1, 2 and 10 to 21 and 40-42, having at least 80, 85, 90, 95, 96, 97, 98, or at least 99% identity with one of these sequences and in which the residue corresponding to the tyrosine at position 135 of SEQ ID NO: 1 is substituted, preferably by a phenylalanine, as presented in SEQ ID NO: 2. In particular, the Spo11 domain may be a variant of a Spo11 protein, said variant comprising a sequence having at least 80%, 85%, 90%, 95%, 96%, 97%, 98%, or at least 99% identity with one of the sequences SEQ ID NO: 1, 10 to 21 and 40-42 and in which the residue corresponding to the tyrosine at position 135 of SEQ ID NO: 1 is substituted, preferably by a phenylalanine. According to one preferred embodiment, the Spo11 domain may be a variant of a Spo11 protein, said variant comprising a sequence having at least 80%, 85%, 90%, 95%, 96%, 97%, 98%, or at least 99% identity with one of the sequences of SEQ ID NO: 10 to 21 and 40-42 and in which the residue corresponding to the tyrosine at position 135 of SEQ ID NO: 1 is substituted, preferably by a phenylalanine.

Preferably, the Spo11 domain with deficient nuclease activity is still capable of recruiting one or more of the partners of Spo11, in particular one or more of the partners described below. Thus, although the nuclease activity of the Spo11 domain may be impaired, its ability to interact with Spo11 partners is preferably retained.

Alternatively, the Spo11 domain of the fusion protein can be replaced by one of the Spo11 partners involved in the formation and repair of double-strand breaks during meiosis. In particular, the Spo11 partner as used in the fusion protein is capable of recruiting Spo11, preferably is a protein that forms a complex with Spo11 and thereby induces the formation of double-strand breaks or their repair. This partner can be selected from the proteins cited in the articles by Keeney et al (2001 Curr. Top. Dev. Biol, 52, pp 1-53), Smith et al (Curr. Opin. Genet. Dev, 1998, 8, pp 200-211), Acquaviva et al (2013 Science, 339, pp 215-8), Vrielynck et al, (2016 Science, 351, pp 939-943), Roberts et al (Science 2016: Vol. 351, Issue 6276, pp 943-949) and Frank R. Blattner (Plant Systematics and Evolution volume 302, pages 239-244(2016). Preferably, the fusion protein according to the invention comprises a Spo11 partner selected from Rec102, Rec103/Sk18, Rec104, Rec114, MTOPOVIB, Mer1, Mer2/Rec107, Mei4, Mre2/Nam8, Mre11, Rad50, Xrs2/Nbs1, Hop1, Red1, Mek1, Set1, Ski8, and Spp1, and variants and orthologues thereof. Preferably, the partner replacing the Spo11 domain comprises a protein selected from Mei4, Mer2, Rec102, Rec104, Rec 14, Set1, Spp1, and MTOPVIB, and variants and orthologues thereof. As contemplated herein, variants of these proteins have at least 80, 85, 90, 95, 96, 97, 98, or at least 99% sequence identity with one of these proteins and are capable of recruiting Spo11. Most preferably, the Spo11 partner is a topoisomerase, preferably selected from the TOPOVIB family and one of its variants and orthologues, more preferably a meiosis-active MTOPOVIB or MTOPOVIBL (Vrielynck, et al, (2016 Science 351 pp 939-943).

All the embodiments described for the fusion protein with a Spo11 domain which is a Spo11 protein or a variant thereof also apply to fusion proteins in which the Spo11 domain is one of the Spo11 partners.

The fusion protein according to the present invention also comprises a domain that is a nuclease associated with a CRISPR system (CRISPR domain). The CRISPR domain is the domain of the fusion protein which is capable of interacting with the guide RNA or RNAs and of targeting the activity of the fusion protein to a given chromosomal region.

According to an embodiment, the fusion protein according to the invention comprises a nuclease associated with a class II CRISPR system, preferably type II, V or VI, more preferably type V or type VI, preferentially type V.

According to an embodiment, the fusion protein according to the invention comprises a nuclease associated with a class II and type II CRISPR system, in particular excluding a Cas9-type nuclease, for example Cns2, in particular the Cns2 nuclease of Streptococcus thermophilus, for example as described under GenBank accession number: AEM62890.1, of Streptococcus pyogenes, for example as described under GenBank accession number: ANC25453.1, or of Streptococcus canis for example as described under GenBank accession number: VTR80107.1. Preferably, the nuclease associated with a CRISPR system is not a Cas9 nuclease. According to an embodiment, the fusion protein according to the invention comprises a nuclease associated with a class II and type VI CRISPR system, preferably Cas13a (C2c2), Cas13b or Cas13c, for example the Cas13a nuclease of Herbinix hemicellulosilytica, in particular as described under GenBank accession number: WP_103203632.1, the Cas13a nuclease of Lachnospiraceae bacterium, in particular as described under GenBank accession number: WP_022785443.1, or the Cas13a nuclease of Leptotrichia wadei, in particular as described under GenBank accession number: WP_021746003.1.

According to an embodiment, the fusion protein according to the invention comprises a nuclease associated with a class II and type V CRISPR system, preferably Cpf1 (also called Cas12a), C2c1 (also called Cas12b) or C2c3.

In a preferred embodiment, the fusion protein comprises a class II CRISPR domain, preferably of type V and preferentially a Cpf1 domain.

The nucleases associated with a CRISPR system as contemplated may comprise, or consist of, a nuclease selected from any of the nucleases mentioned above, and variants thereof comprising a sequence having at least 80, 85, 90, 95, 96, 97, 98, or at least 99% identity with one of these sequences, and a CRISPR system-associated nuclease activity. As used herein, the term ‘CRISPR system-associated nuclease activity’ refers to a nuclease activity and/or the ability to interact with the guide RNA and recognize the targeted region of the nucleic acid.

According to a particular embodiment, the nuclease associated with a CRISPR system is a Cpf1 nuclease, a variant or a fragment thereof capable of interacting with the guide RNAs. According to a preferred embodiment, the nuclease associated with a CRISPR system is a Cpf1 nuclease or a variant thereof.

The Cpf1 nuclease can be selected from Cpf1 proteins from bacteria of the genera Prevotella, Moraxella, Leptospira, Lachnospiraceae, Francissela, Candidatus, Eubacterium, Parcubacteria, Peregrinibacteria, Acidmicococcus and Prophyromonas. In particular, the Cpf1 domain can be selected from the Cpf1 proteins of Parcubacteria bacterium GWC2011_GWC2_44_17 (PbCpf1, for example as described under GenBank accession number KKT48220.1, SEQ ID NO: 22), of Peregrinibacteria bacterium GW2011_GWA_33_10 (PeCpf1, for example as described under GenBank accession number KKP36646.1, SEQ ID NO: 23), of Acidaminococcus sp. BVBLG (AsCpf1, for example as described under GenBank accession number WP_021736722.1, SEQ ID NO: 24), of Prophyromonas macacae (PmCpf1, for example as described under GenBank accession number WP_018359861.1, SEQ ID NO: 25), of Prophyromonas crevioricanis (PcCpf1, for example as described under GenBank accession number WP_036890108.1, SEQ ID NO: 26), of Francisella tularensis (UniProtKB: AOQ7Q2, SEQ ID NO: 27), of Acidaminococcus sp. (UniProtKB: U2UMQ6, SEQ ID NO: 28), of Prevotella disiens (PdCpF1, for example as described under GenBank accession number WP_004356401.1, SEQ ID NO: 29), of Moraxella bovoculi 237 (MbCpf1, for example as described under GenBank accession number KDN25524.1, SEQ ID NO: 30), of Leptospira inadai (LiCpf1, for example as described under GenBank accession number WP_020988726.1, SEQ ID NO: 31), of Lachnospiraceae bacterium MA2020 (LbCpf1, for example as described under GenBank accession number WP_044919442.1, SEQ ID NO: 32), of Francisella novicida U112 (FnCpf1, for example as described under GenBank accession number WP_003040289.1, SEQ ID NO: 33). Of course, these examples are not limiting and any known Cpf1 protein can be used in the fusion protein or process according to the invention.

According to an embodiment, the Cpf1 domain comprises, or consists of, a Cpf1 protein selected from the wild-type Cpf1 proteins, variants and fragments thereof having Cpf1 activity. Preferably, said variants have at least 80, 85, 90, 95, 96, 97, 98, or at least 99% identity with one of these Cpf1 proteins.

According to a particular embodiment, the Cpf1 domain comprises, or consists of, a protein comprising a sequence selected from the sequences described above, in particular selected from SEQ ID NO: 22 to 33 and variants thereof having at least 80, 85, 90, 95, 96, 97, 98, or at least 99% identity with one of these sequences and Cpf1 activity. As used herein, the term ‘Cpf1 activity’ refers to nuclease activity and/or the ability to interact with the guide RNA and recognize the targeted region of the nucleic acid, preferably the ability to interact with the guide RNA and recognize the targeted region of the nucleic acid and optionally nuclease activity, in particular endonuclease DNA activity. The ability of a protein to interact with the guide RNA and to recognize the targeted region of the nucleic acid may readily be tested by the person skilled in the art, especially by conventional techniques such as chromatin immunoprecipitation (ChIP) with an antibody recognizing the protein and its location on the DNA by PCR or sequencing. The nuclease activity, and in particular the endonuclease DNA activity, may readily be tested by the person skilled in the art, especially by conventional techniques such as Southern blot DNA hybridization or by using the technique described in the article by Zetsche, et al. (2015) Cell, 163, 759-771.

For a wild-type Cpf1 protein or a variant endowed with nuclease activity, the term “Cpf1 activity” refers preferably to a nuclease activity, in particular endonuclease DNA activity, and to the ability to interact with the guide RNA and to recognize the targeted region of the nucleic acid. For a Cpf1 variant which exhibits deficient nuclease activity, the term “Cpf1 activity” refers preferably to the ability to interact with the guide RNA and to recognize the targeted region of the nucleic acid.

According to a particular aspect, the fusion protein comprises a variant or mutant of Cpf1. For example, the fusion protein according to the invention may comprise a Cpf1 domain with abolished, reduced or enhanced nuclease activity. In particular, this type of mutant comprises mutations in the RuvC nuclease domain of Cpf1. To facilitate targeting to different regions of the genome, the Cpf1 domain may also include mutations altering PAM recognition as described in Gao et al, Nat Biotechnol. 2017 August; 35(8): 789-792. The Cpf1 protein can also be truncated to remove domains of the protein that are non-essential to the functions of the fusion protein, particularly domains of the Cpf1 protein that are not required for interaction with the guide RNA.

According to a particular embodiment, the Cpf1 domain comprises, or consists of, the sequence of FnCpf1 (SEQ ID NO: 3), or LbCpf1 (SEQ ID NO: 4) or a sequence having at least 80, 85, 90, 95, 96, 97, 98, or at least 99% identity with one of these sequences. According to one particular embodiment, the Cpf1 domain comprises, or consists of, a protein comprising a sequence selected from the sequences described above, in particular selected from the sequences SEQ ID NO: 3, 4 and 22 to 33, and the variants and fragments of said sequences having Cpf1 activity. Said variants preferably have at least 80, 85, 90, 95, 96, 97, 98, or at least 99% identity with one of these Cpf1 proteins.

According to another particular embodiment, the Cpf1 domain comprises, or consists of, a protein comprising a sequence selected from the sequences described above, in particular selected from the sequences SEQ ID NO: 3, 4 and 22 to 33 and the variants of said sequences comprising a sequence having at least 80, 85, 90, 95, 96, 97, 98, or at least 99% identity with one of these sequences and a Cpf1 activity.

Alternatively, the domain that is a nuclease associated with a CRISPR system may exhibit deficient nuclease activity. ‘Deficient nuclease activity’ means in particular a reduced, diminished or non-existent nuclease activity, in particular relative to the nuclease activity of the wild-type protein. The nuclease associated with a CRISPR system preferably has a reduced or suppressed endonuclease DNA activity relative to the nuclease activity of the wild-type protein. The nuclease associated with a CRISPR system which has deficient nuclease activity retains its ability to interact with a guide RNA and therefore still enables the targeting of the fusion protein to a given chromosomal region.

According to a particular embodiment, the DNA or RNA cleavage capacity and/or hydrolase activity of the nuclease is reduced or diminished, in particular relative to the nuclease activity of the wild-type protein. Preferably, the nuclease activity of the protein associated with a CRISPR system with deficient nuclease activity is reduced by at least 50, 55, 60, 65, 70, 75, 80, 85, 90, 95 or 100%, relative to the nuclease activity of the wild-type protein.

In particular, the nuclease associated with a CRISPR system, preferably class II and type V, with deficient nuclease activity may comprise a mutated catalytic site, the mutation negatively impacting the nuclease or hydrolytic capacity of the protein.

Preferably, the CRISPR system-associated nuclease deficient in nuclease activity may comprise, or consist of, a mutant Cpf1 protein for example as described in Zhang et al, Cell Discov. 2018; 4: 36, comprising the D832A mutation. A Cpf1 protein with such a substitution is unable to induce DNA double-strand breaks, and may in particular be referred to as ‘dead Cpf1’ or ‘dCpf1’. In particular, the Cpf1 domain can be a variant of a Cpf1 protein, preferably of a wild-type Cpf1, having at least 80, 85, 90, 95, 96, 97, 98, or at least 99% identity with said Cpf1 protein and in which the residue corresponding to the aspartate at position 832 of SEQ ID NO: 4 is substituted, preferably by an alanine. More particularly, the Cpf1 domain may be a variant of the sequences SEQ ID NO: 3, 4 and 22 to 33 having at least 80, 85, 90, 95, 96, 97, 98, or at least 99% identity with one of these sequences and in which the residue corresponding to the aspartate at position 832 of SEQ ID NO: 4 is substituted, preferably by an alanine. Preferably, the dCpf1 domain may comprise or consist of the sequence SEQ ID NO: 5 or SEQ ID NO: 6. The residue corresponding to the aspartate at position 832 of SEQ ID NO: 4 in the sequence of a Cpf1 protein may be easily identified by conventional sequence alignment techniques.

The fusion protein preferably comprises at least one domain, CRISPR or Spo11, with nuclease activity. According to an embodiment, the fusion protein comprises a Spo11 domain with nuclease activity and a domain that is a nuclease associated with a CRISPR system, preferably a Cpf1 nuclease, deficient in nuclease activity. According to a preferred embodiment, the fusion protein comprises a Spo11 domain deficient in nuclease activity and a domain that is a nuclease associated with a CRISPR system, preferably a Cpf1 nuclease, with nuclease activity.

Alternatively, the fusion protein comprises (i) a Spo11 domain with deficient nuclease activity and (ii) a CRISPR domain with deficient nuclease activity. Thus, the fusion protein of the present invention may comprise a domain that is a nuclease associated with a CRISPR system and a Spo11 domain, both domains with deficient nuclease activity.

According to an embodiment, the Spo11 domain is on the N-terminal side and the CRISPR domain, preferably the Cpf1 domain, on the C-terminal side of the fusion protein. According to another embodiment, the Spo11 domain is on the C-terminal side and the CRISPR domain, preferably the Cpf1 domain, on the N-terminal side of the fusion protein.

The fusion protein may also comprise a nuclear localization signal (NLS) sequence. NLS sequences are well known to the skilled person and generally comprise a short sequence of basic amino acids. By way of example, the NLS sequence may comprise the sequence PKKKRKV (SEQ ID NO: 7). The NLS sequence may be present at the N-terminus, the C-terminus or in an internal region of the fusion protein.

The fusion protein may also comprise an additional cell-penetration domain, i.e. a domain facilitating the entry of the fusion protein into the cell. This type of domain is well known to the skilled person and may comprise, for example, a penetration peptide sequence derived from the HIV-1 TAT protein such as GRKKRRQRRRPPQPKKKRKV (SEQ ID NO: 8), derived from the TLM sequence of the human hepatitis B virus such as PLSSIFSRIGDPPKKKRKV (SEQ ID NO: 9), or a polyarginine peptide sequence. This cell-penetration domain may be present at the N-terminus, C-terminus, or within the fusion protein.

The fusion protein may further comprise one or more linkage sequences (linkers) between the CRISPR domain, in particular class II, preferably type V and preferentially Cpf1, and the Spo11 domain, and optionally between these domains and other domains of the protein such as the nuclear localization signal sequence or the cell-penetration domain. The length of these linkers is easily adjustable by the skilled person. In general, these sequences comprise between 10 and 20 amino acids, preferably about 15 amino acids and even more preferably 12 amino acids. The linkers between the different domains can be of identical or different lengths.

According to a particular embodiment, the fusion protein comprises, or consists of, successively, from the N-terminus to the C-terminus: a nuclear localization signal, a first linker (linker1), a CRISPR domain, preferably a Cpf1 domain, a second linker (linker2) and a Spo11 domain.

According to another particular embodiment, the fusion protein comprises, or consists of, successively, from the N-terminus to the C-terminus: a nuclear localization signal, a first linker (linker1), a Spo11 domain, a second linker (linker2) and a CRISPR domain, preferably a Cpf1 domain.

The fusion protein may further comprise a label (or a tag) that is a defined sequence of amino acids. This tag can in particular be used to detect the expression of the fusion protein, to identify proteins interacting with the fusion protein or to characterize the binding sites of the fusion protein in the genome. The detection of the tag attached to the fusion protein can be performed with an antibody specific for said tag or any other technique well known to the skilled person. Identification of the proteins interacting with the fusion protein can be carried out, for example, by co-immunoprecipitation techniques. The characterization of the binding sites of the fusion protein in the genome can be carried out, for example, by immunoprecipitation, chromatin immunoprecipitation coupled with real-time quantitative PCR (ChIP-qPCR), chromatin immunoprecipitation coupled with sequencing (ChIP-Seq), oligonucleotide mapping or any other technique well known to the skilled person.

This tag may be present at the N-terminus of the fusion protein, at the C-terminus of the fusion protein, or at a non-terminal position in the fusion protein. Preferably, the tag is present at the C-terminus of the fusion protein. The fusion protein may comprise one or more tags, identical or different and in any combination of localization, in particular at the N-terminus, C-terminus, N- and C-termini or at positions internal to the fusion protein.

The tags, as used in the present invention, may be selected from the many tags well known to the skilled person. In particular, the tags used in the present invention may be peptide tags and/or protein tags. Preferably, the tags used in the present invention are peptide tags. Examples of peptide tags that can be used in the present invention include, but are not limited to, tags formed from repeats of at least six histidines (His), in particular tags formed from six or eight histidines, as well as the Flag, polyglutamate, hemagglutinin (HA), calmodulin, Strep, E-tag, myc, V5, Xpress, VSV, S-tag, Avi, SBP, Softag 1, Softag 2, Softag 3, isopetag, SpyTag, and tetracysteine tags, and combinations thereof. Examples of protein tags that can be used in the present invention include, but are not limited to, Glutathione-S-Transferase (GST), Staphylococcus aureus protein A, Nus A, chitin binding protein (CBP), thioredoxin, maltose binding protein (MBP), biotin carboxyl carrier protein (BCCP), immunoglobulin constant fragment (Fc), tags comprising a fluorescent protein such as GFP (Green Fluorescent Protein), RFP (Red Fluorescent Protein), CFP (Cyan Fluorescent Protein) or YFP (Yellow Fluorescent Protein), and combinations thereof.

According to a preferred embodiment, the fusion protein comprises a tag formed by six histidines and/or one or more Flag motifs, preferably three Flag motifs. According to an embodiment, the fusion protein comprises a tag formed by three Flag motifs followed by 6 histidines at the C-terminus of the fusion protein. Additionally or alternatively, the fusion protein comprises a V5 motif, preferably on the N-terminal side of the fusion protein. The V5 tag is derived from a small epitope (Pk) found on the P and V proteins of simian virus family 5 (SV5) paramyxovirus. The V5 tag is generally used with 14 amino acids (GKPIPNPLLGLDST, SEQ ID NO 38) or 9 amino acids shorter (IPNPLLGLD, SEQ ID NO: 39). According to a particular embodiment, the fusion protein comprises a tag formed by six histidines and three Flag motifs, preferably C-terminal, and an N-terminal V5 motif.

The fusion protein as described above can be introduced into the cell in a protein form, in particular in its mature form or in the form of a precursor, preferably in its mature form, or in the form of a nucleic acid encoding said protein.

When the fusion protein is introduced into the cell in a protein form, protecting groups can be added to the C- and/or N-termini to improve the resistance of the fusion protein to peptidases. For example, the protective group at the N-terminus may be acylation or acetylation and the protective group at the C-terminus may be amidation or esterification. The action of proteases can also be counteracted by the use of D-configuration amino acids, cyclization of the protein by formation of disulphide bridges, lactam rings or linkages between the N- and C-termini. The fusion protein of the invention may also comprise pseudo-peptide bonds replacing the ‘classical’ CONH peptide bonds and conferring increased resistance to peptidases, such as CHOH—CH2, NHCO, CH2-O, CH2CH2, CO—CH2, N—N, CH═CH, CH2NH, and CH2-S. The fusion protein may also comprise one or more amino acids which are rare amino acids, in particular hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid; or synthetic amino acids, in particular ornithine, norleucine, norvaline, and cyclohexyl-alanine.

The fusion protein according to the invention can be obtained by conventional chemical synthesis (in solid phase or in liquid homogeneous phase) or by enzymatic synthesis (Kullmann W, Enzymatic peptide synthesis, 1987, CRC Press, Florida). It can also be obtained by a method consisting in culturing a host cell expressing a nucleic acid encoding the fusion protein and recovering said protein from these cells or from the culture medium.

The present invention also relates to a nucleic acid encoding the fusion protein according to the invention, in particular a fusion protein comprising a class II nuclease of the CRISPR system, preferably type V and preferentially Cpf1, and a Spo11 or Spo11 partner protein as described above.

For the purposes of the invention, ‘nucleic acid’ means any DNA- or RNA-based molecule. These molecules may be synthetic or semi-synthetic, recombinant, possibly amplified or cloned into vectors, chemically modified, comprising non-natural bases or modified nucleotides comprising for example a modified bond, a modified purine or pyrimidine base, or a modified sugar. Preferably, the use of codons is optimized according to the nature of the eukaryotic cell of interest.

The nucleic acid according to the invention may be in the form of DNA and/or RNA, single-stranded or double-stranded. According to a preferred embodiment, the nucleic acid is an isolated DNA molecule, synthesized by recombinant techniques well known to the person skilled in the art. The nucleic acid according to the invention can be deduced from the sequence of the fusion protein according to the invention and the use of codons can be adapted according to the host cell in which the nucleic acid is to be transcribed.

The present invention further relates to an expression cassette comprising a nucleic acid according to the invention operably linked to the sequences necessary for its expression. In particular, the nucleic acid may be under the control of a promoter enabling its expression in a eukaryotic host cell. In general, an expression cassette comprises, or consists of, a promoter for initiating transcription, a nucleic acid according to the invention, and a transcription terminator.

The term ‘expression cassette’ refers to a nucleic acid construct comprising a coding region and a regulatory region, operably linked. The term ‘operably linked’ indicates that the elements are combined in such a way that the expression of the coding sequence is under the control of the transcriptional promoter. Typically, the promoter sequence is placed upstream of the gene of interest, at a distance from it compatible with control of its expression. Spacer sequences may be present between the regulatory elements and the gene, as long as they do not prevent expression. The expression cassette may also include at least one activating ‘enhancer’ sequence operably linked to the promoter.

A wide variety of promoters that can be used for the expression of genes of interest in cells or host organisms are available to the skilled person. They include constitutive promoters as well as inducible promoters that are activated or repressed by exogenous physical or chemical stimuli.

Preferably, the nucleic acid according to the invention is placed under the control of a constitutive promoter or a meiosis-specific promoter.

Examples of meiosis-specific promoters that can be used in the present invention include, but are not limited to, endogenous Spo11 promoters, promoters of Spo11 partners for double-strand break formation, the Rec8 promoter (Murakami & Nicolas, 2009, Mol. Cell. Biol, 29, 3500-16), or the Spo13 promoter (Malkova et al, 1996, Genetics, 143, 741-754), meiotic promoters from Arabidopsis thaliana for example as described in Li et al, BMC Plant Biol. 2012; 12: 104, Eid et al, Plant cell Rep. 2016 July; 35(7):1555-8, Xu et al, Front. Plant Sci., 13 Jul. 2018e, Da Ines et al, PLoS Genet, 2013, 9, e1003787).

Other inducible promoters can also be used such as the oestradiol promoter (Carlie & 15 Amon, 2008 Cell, 133, 280-91), the methionine promoter (Care et al, 1999, Molecular Microb 34, 792-798), the doxycycline-inducible TetO/TetR system, heat shock-induced promoters, metals, steroids, antibiotics, and alcohol.

Constitutive promoters that can be used in the context of the present invention are, by way of non-limiting examples: cytomegalovirus (CMV) immediate-early gene promoter, simian virus (SV40) promoter, adenovirus major late promoter, Rous sarcoma virus (RSV) promoter, mouse mammary tumor virus (MMTV) promoter, phosphoglycerate kinase (PGK) promoter, ED1-alpha elongation factor promoter, ubiquitin promoters, actin promoters, tubulin promoters, immunoglobulin promoters, alcohol dehydrogenase 1 (ADH1) promoter, RNA polymerase III-dependent promoters such as the U6, U3, H1, 7SL, pRPR1 (‘Ribonuclease P RNA 1’), and SNR52 (‘small nuclear RNA 52’) promoters, or the pZmUbi promoter.

Preferably, the expression cassette and/or the nucleic acid according to the invention is operably linked to a transcriptional promoter allowing expression of the expression cassette and/or the nucleic acid during meiosis. For example, such a promoter may be pREC8.

The transcription terminator can be easily chosen by the skilled person. Preferably, this terminator is RPR1t, the 3′ flanking sequence of the SUP4 gene of Saccharomyces cerevisiae or the nopaline synthase terminator tNOS.

The present invention further relates to an expression vector comprising a nucleic acid or an expression cassette according to the invention. This expression vector can be used to transform a host cell and allow expression of the nucleic acid according to the invention in said cell. The vectors can be constructed by conventional molecular biology techniques, well known to the skilled person.

Advantageously, the expression vector comprises regulatory elements allowing the expression of the nucleic acid according to the invention. These elements may comprise, for example, transcription promoters, transcription activators, terminator sequences, and initiation and termination codons. Methods for selecting these elements according to the host cell in which expression is desired are well known to the skilled person.

In a particular embodiment, the expression vector comprises a nucleic acid encoding the fusion protein according to the invention, placed under the control of a constitutive promoter, preferably the ADH1 promoter (pADH1). It may also comprise a terminator sequence such as the ADH1 terminator (tADH1).

The expression vector may comprise one or more bacterial or eukaryotic origins of replication. In particular, the expression vector may comprise a bacterial origin of replication functional in E. coli such as the ColE1 origin of replication. Alternatively, the vector can comprise a eukaryotic origin of replication, preferably functional in plants and in yeast, in particular in S. cerevisiae.

The vector may further comprise elements allowing its selection in a bacterial or eukaryotic host cell such as, for example, an antibiotic resistance gene or a selection gene ensuring the complementation of the respective inactivated gene in the host cell genome. Such elements are well known to the skilled person and widely described in the literature.

In a particular embodiment, the expression vector comprises one or more antibiotic resistance genes, preferably an ampicillin, kanamycin, hygromycin, geneticin and/or nourseothricin resistance gene.

The expression vector may also comprise one or more sequences allowing targeted insertion of the vector, expression cassette, or nucleic acid into the genome of a host cell. Preferably, the insertion is performed at a gene whose inactivation allows the selection of host cells that have integrated the vector, cassette, or nucleic acid, such as the TRP1 locus.

The vector can be circular or linear, single- or double-stranded. It is advantageously selected from plasmids, phages, phagemids, viruses, cosmids, and artificial chromosomes. Preferably, the vector is a plasmid.

The present invention relates in particular to a vector, preferably a plasmid, comprising a bacterial origin of replication, preferably the ColE1 origin, a nucleic acid as defined above under the control of a promoter, preferably a constitutive promoter such as the ADH1 promoter, a terminator, preferably the ADH1 terminator, one or more selection markers, preferably resistance markers such as the kanamycin or ampicillin resistance gene, and one or more sequences allowing the targeted insertion of the vector, expression cassette, or nucleic acid into the host cell genome, preferably at the TRP1 locus of the yeast genome.

According to an embodiment, the invention relates in particular to a vector, preferably a plasmid, compatible for agrotransfection, in particular using Agrobacterium tumefaciens (Fraley et al Crit. Rev. Plant. Sci. 4 pp 1-46; Fromm et al, (1990) Biotechnology 8, pp 833-844) or Agrobacterium rhizogenes (Cho et al (2000) Planta 210 pp 195-204) and aimed at plant transformation. An example of a compatible plasmid is the Ti type plasmid, in particular pBIN19 (Lee and Gelvin (2008) Plant Physiology 146(2) pp 325-332). In particular, when intended for use in planta, the vector according to the invention comprises a selectable marker. The most commonly used selectable markers for plant transformation include the kanamycin neomycin phosphotransferase II (NPTII) resistance conferring gene, used for selection in a kanamycin-containing culture, the phosphinothricin acetyltransferase (HPH) gene, used for selection in a culture containing hygromycin B.

In a particular embodiment, the nucleic acid according to the invention carried by the vector encodes a fusion protein comprising one or more tags, preferably comprising a tag consisting of six histidines and/or one or more Flag motifs, preferably three Flag motifs. Preferably the tag or tags are C-terminal.

The present invention also relates to the use of a nucleic acid, an expression cassette or an expression vector according to the invention to transform or transfect a cell. The host cell may be transiently or stably transformed/transfected and the nucleic acid, cassette or vector may be contained within the cell as an episome or integrated into the host cell genome.

The present invention thus relates to a host cell comprising a fusion protein, a nucleic acid, a cassette or an expression vector according to the invention.

Preferably, the cell is a eukaryotic cell.

As used herein, the term ‘eukaryotic cell’ refers to a yeast cell, a plant cell, a fungal cell or an animal cell, in particular a mammalian cell such as a mouse or rat cell, or an insect cell. The eukaryotic cell is preferably non-human and/or non-embryonic. Preferably the eukaryotic cell is not an embryonic stem cell of human and/or animal origin.

According to preferred embodiments, the eukaryotic cell expresses a Spo11 protein endowed with nuclease activity, i.e. a Spo11 protein capable of inducing double-strand breaks during prophase I of meiosis. This protein can be an endogenous Spo11 protein of the cell or a heterologous Spo11 protein. Preferably, this protein is an endogenous Spo11 protein of the cell.

According to a particular embodiment, the eukaryotic cell is a yeast cell, in particular a yeast of industrial interest. Examples of yeasts of interest include, but are not limited to, yeasts of the genus Saccharomyces sensu stricto, Schizosaccharomyces, Yarrowia, Hansenula, Kluyveromyces, Pichia or Candida, as well as hybrids obtained from a strain belonging to one of these genera.

Preferably, the yeast of interest belongs to the genus Saccharomyces, preferably a yeast selected from the group consisting of Saccharomyces cerevisiae, Saccharomyces bayanus, Saccharomyces castelli, Saccharomyces eubayanus, Saccharomyces kluyveri, Saccharomyces kudriavzevii, Saccharomyces mikatae, Saccharomyces uvarum, Saccharomyces paradoxus, Saccharomyces pastorianus (also called Saccharomyces carlsbergensis), and hybrids obtained from at least one strain belonging to one of these species such as for example an S. cerevisiae S. paradoxus hybrid or an S. cerevisiae S. uvarum hybrid, more preferably said eukaryotic host cell is Saccharomyces cerevisiae.

According to another particular embodiment, the eukaryotic cell is a fungal cell, in particular a fungal cell of industrial interest. Examples of fungi include, but are not limited to, cells of filamentous fungi. Filamentous fungi include fungi belonging to the subdivisions Eumycota and Oomycota. The cells of filamentous fungi may be selected from the group consisting of cells of Trichoderma, Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Sordaria, Talaromyces, Thermoascus, Thielavia, Tolypocladium or Trametes.

According to yet another particular embodiment, the eukaryotic cell is a plant cell, in particular a plant cell of agronomic, horticultural, pharmaceutical or cosmetic interest, in particular vegetables, fruits, herbs, flowers, trees and shrubs.

The plant cell is preferably selected from monocotyledonous plants and dicotyledonous plants, more particularly preferably selected from the group consisting of rice, wheat, soybean, maize, tomato, onion, cucumber, lettuce, asparagus, carrot, turnip, Arabidopsis thaliana, barley, rapeseed, cotton, grapevine, sugarcane, beet, cotton, sunflower, oil palm, coffee, tea, cocoa, chicory, bell pepper, chili, lemon, orange, nectarine, mango, apple, banana, peach, apricot, sweet potato, yam, almond, hazelnut, strawberry, melon, watermelon, olive tree, potato, zucchini, eggplant, avocado, cabbage, plum, cherry, pineapple, spinach, apple, mandarin, grapefruit, pear, grape, clove, cashew nut, coconut, sesame, rye, hemp, tobacco, berries such as raspberry or blackcurrant, peanut, castor, vanilla, poplar, eucalyptus, green foxtail, cassava, and horticultural plants such as, for example, roses, tulips, orchids and geraniums. In particular, the plant cell may be selected from the group consisting of rice, wheat, soybean, maize, tomato, onion, cucumber, lettuce, asparagus, carrot, turnip, Arabidopsis thaliana, barley, rapeseed, cotton, grapevine, sugarcane, beet, cotton, sunflower, palm olive tree, coffee, tea, cocoa, chicory, bell pepper, chili, lemon, orange, nectarine, mango, apple, banana, peach, apricot, sweet potato, yam, almond, hazelnut, strawberry, melon, watermelon, olive tree, and horticultural plants such as roses, tulips, orchids and geraniums.

Each of the different cells described above can in particular be used in the processes according to the invention described below.

Thus, the present invention also relates to the use of the fusion protein, the nucleic acid, the expression cassette or the expression vector according to the invention to (i) induce targeted meiotic recombinations in a eukaryotic cell, (ii) generate variants of a eukaryotic organism, and/or (iii) identify or locate genetic information encoding a trait of interest in a eukaryotic cell genome.

The invention also relates to processes for (i) inducing targeted meiotic recombinations in a eukaryotic cell, (ii) generating variants of a eukaryotic organism, and/or (iii) identifying or locating genetic information encoding a trait of interest in a eukaryotic cell genome as described below.

The processes according to the invention can be in vitro, in vivo or ex vivo processes, preferably in vitro processes.

The invention relates in particular to a process for inducing targeted meiotic recombinations in a eukaryotic cell, preferably non-human, comprising
introduction into said cell of:
a) a fusion protein, a nucleic acid, an expression cassette, or a vector as described above;
and
b) one or more guide RNAs or one or more nucleic acids encoding said guide RNAs, said guide RNAs comprising a nuclease binding RNA structure associated with a CRISPR system of the fusion protein and a sequence complementary to the targeted chromosomal region; and
induction of entry into prophase I of meiosis of said cell.

In particular, the eukaryotic cell is as described above.

As used in the present application, the term ‘guide RNA’ or ‘gRNA’ refers to an RNA molecule capable of interacting with the CRISPR domain, preferably a Cpf1 protein, of the fusion protein in order to guide it to a target chromosomal region.

Each gRNA comprises a region (commonly called the ‘SDS’ region), at the 3′ end of the gRNA, that is complementary to the target chromosomal region and mimics the crRNA of the endogenous CRISPR system. Unlike other systems such as CRISPR-Cas9, Cpf1 gRNA does not require a second region (commonly called the ‘handle’ region), at the 3′ end of the gRNA, which mimics the base-pairing interactions between the tracrRNA and the crRNA of the endogenous CRISPR system.

The sequence of the gRNA varies according to the targeted chromosomal sequence. In particular, the ‘SDS’ region of the gRNA, which is complementary to the target chromosomal region, generally comprises between 10 and 25 nucleotides. Preferably, this region has a length of 19, 20 or 21 nucleotides, and particularly preferably 25 nucleotides.

The total length of a gRNA is generally 30 to 140 nucleotides, preferably 30 to 125 nucleotides, and more preferably 40 to 100 nucleotides. According to a particular embodiment, a gRNA as used in the present invention has a length of 30 to 75 nucleotides, preferably 35 to 65 nucleotides, more preferably 44 to 63 nucleotides.

The person skilled in the art can easily define the sequence and structure of the gRNAs according to the chromosomal region to be targeted using well known techniques.

In the process according to the invention, one or more gRNAs may be used simultaneously.

These gRNAs may be different or identical. These gRNAs may target identical or different, preferably different, chromosomal regions.

The gRNAs can be introduced into the eukaryotic cell in the form of mature gRNA molecules, in the form of precursors or in the form of one or more nucleic acids encoding said gRNAs.

When the gRNA(s) are introduced into the cell directly in the form of RNA molecules (mature or precursor), these gRNAs may contain modified nucleotides or chemical modifications allowing them, for example, to increase their resistance to nucleases and thus to increase their life span in the cell. In particular, they may comprise at least one modified or unnatural nucleotide such as, for example, a nucleotide comprising a modified base, such as inosine, methyl-5-deoxycytidine, dimethylamino-5-deoxyuridine, deoxyuridine, diamino-2,6-purine, bromo-5-deoxyuridine or any other modified base allowing hybridization. The gRNAs used according to the invention can also be modified at the internucleotide linkage such as phosphorothioates, H-phosphonates or alkyl phosphonates, or at the backbone such as alpha-oligonucleotides, 2′-O-alkyl ribose or peptide nucleic acids (PNAs) (Egholm et al, 1992 J. Am. Chem. Soc., 114, pp 1895-1897).

The gRNAs can be natural RNAs, synthetic RNAs, or RNAs produced by recombinant techniques. These gRNAs can be prepared by any methods known to the skilled person such as, for example, chemical synthesis, in vivo transcription, or amplification techniques.

According to an embodiment, the process comprises the introduction into the eukaryotic cell of the fusion protein and one or more gRNAs capable of targeting the action of the fusion protein to a given chromosomal region. The protein and the gRNAs can be introduced into the cytoplasm or nucleus of the eukaryotic cell by any method known to the skilled person, for example by microinjection. In particular, the fusion protein can be introduced into the cell as an element of a protein-RNA complex comprising at least one gRNA.

According to another embodiment, the process comprises the introduction into the eukaryotic cell of the fusion protein and one or more nucleic acids encoding one or more gRNAs.

According to yet another embodiment, the process comprises the introduction into the eukaryotic cell of a nucleic acid encoding the fusion protein and one or more gRNAs.

According to yet another embodiment, the process comprises the introduction into the eukaryotic cell of a nucleic acid encoding the fusion protein and one or more nucleic acids encoding one or more gRNAs.

According to an embodiment, the eukaryotic cell is heterozygous for the gene(s) targeted by the guide RNA(s).

According to another embodiment, the eukaryotic cell is homozygous for the gene(s) targeted by the guide RNA(s).

The fusion protein, or the nucleic acid encoding it, and the gRNA(s), or the nucleic acid(s) encoding them, can be introduced simultaneously or sequentially into the cell.

Plant transformation techniques are well known and described in the technical and scientific literature. These techniques aim at the transformation of plant cells from whole plants, callus, or protoplasts. These techniques include injection or microinjection (Griesbach (1987) Plant Sci. 50 69-77), DNA electroporation (Fromm et al (1985), Natl Acad Sci., USA 82: 5824; Wan and Lemaux, Plant Physiol. 104 (1994), 37-48), biolistics (Klein et al (1987) Nature 327: 773), viral vector transfection (Gelvin, Nature Biotechnology 23, 684-685 (2005)), bombardment (Sood et al, 2011, Biologia Plantarum, 55, 1-15), cell or protoplast fusion (Willmitzer, L., 1993, Transgenic plants. Biotechnology, vol. 2, 627-659), agrotransfection by T-DNA insertion, in particular using Agrobacterium tumefaciens (Fraley et al Crit. Rev. Plant. Sci. 4 1-46; Fromm et al, Biotechnology 8 (1990), 833-844) or Agrobacterium rhizogenes (Cho et al (2000) Planta 210: 195-204) or other bacterial hosts (Brootghaerts et al (2005) Nature 433: 629-633) using for example the floral dip technique (Clough and Bent 1998; Zale et al 2009).

Alternatively, and more particularly concerning plant cells, the nucleic acid encoding the fusion protein and the nucleic acid(s) encoding the gRNA(s) can be introduced into a cell by crossing two cells into which the nucleic acid encoding the fusion protein and the nucleic acid(s) encoding the gRNA(s), respectively, have been introduced.

Alternatively, and more particularly concerning plant cells, the nucleic acid encoding the fusion protein and the nucleic acid(s) encoding the gRNA(s) may be introduced into a cell by mitosis of a cell into which the nucleic acid encoding the fusion protein and the nucleic acid(s) encoding the gRNA(s) have previously been introduced.

In embodiments wherein the fusion protein and/or the gRNA(s) are introduced into the eukaryotic cell in the form of a nucleic acid encoding said protein and/or gRNA(s), expression of said nucleic acids produces the fusion protein and/or the gRNA(s) in the cell.

The nucleic acids encoding the fusion protein and those encoding the gRNAs can be placed under the control of identical or different constitutive or inducible promoters, in particular meiosis-specific promoters. According to a preferred embodiment, the nucleic acids are placed under the control of constitutive promoters such as the ADH1 promoter or the RNA polymerase III-dependent pRPR1 and SNR52 promoters, most preferably the pRPR1 promoter.

The nature of the promoter may also depend on the nature of the eukaryotic cell. According to a particular embodiment, the eukaryotic cell is a plant cell, preferably a rice cell, and the nucleic acids are placed under the control of a promoter selected from the pZmUbi promoters (maize ubiquitin promoter) and the U3 and U6 polymerase III promoters. According to a preferred embodiment, the nucleic acid encoding the fusion protein is placed under the control of the pZmUbi promoter and the nucleic acids encoding the gRNAs are placed under the control of the U3 or U6 promoter, preferably the U3 promoter.

According to a particular aspect, gRNA expression is placed under the control of the tetracycline operator. The Tet system comprises two complementary circuits: the tTA dependent circuit (Tet-Off system) and the rtTA dependent circuit (Tet-On system). Preferably, gRNA expression is controlled by a Tet-On system. gRNA expression is thus regulated by the presence or absence of tetracycline or one of its derivatives such as doxycycline.

The nucleic acids encoding the fusion protein and the gRNA(s) may be arranged on the same construct, in particular on the same expression vector, or on separate constructs. Alternatively, the nucleic acids may be inserted into the eukaryotic cell genome at identical or distinct regions. According to a preferred embodiment, the nucleic acids encoding the fusion protein and the gRNA(s) are arranged on the same expression vector.

The nucleic acids as described above can be introduced into the eukaryotic cell by any method known to the skilled person, in particular by microinjection, transfection, agro-infection, electroporation or biolistics.

Optionally, the expression or activity of the endogenous Spo11 protein of the eukaryotic cell can be suppressed in order to better control meiotic recombination phenomena. This inactivation can be carried out by techniques well known to the skilled person, in particular by inactivating the gene encoding the endogenous Spo11 protein or by inhibiting its expression by means of interfering RNA. Preferably, in this embodiment, the Spo11 domain of the fusion protein is endowed with nuclease activity in order to complement the absence of endogenous Spo11 activity.

After introduction into the eukaryotic cell of the fusion protein and one or more gRNAs, or the nucleic acids encoding them, the process according to the invention comprises the induction of the entry into prophase I of meiosis of said cell.

This induction can be done according to different methods, well known to the skilled person.

By way of example, when the eukaryotic cell is a mouse cell, the entry of cells into prophase I of meiosis can be induced by the addition of retinoic acid (Bowles J et al, 2006, Sciences, 312(5773), pp 596-600).

When the eukaryotic cell is a plant cell, the induction of meiosis occurs according to a natural process. According to a particular embodiment, after transformation of a callus comprising one or more plant cells, a plant is regenerated and placed in conditions favoring the induction of a reproductive phase and thus of the meiosis process. These conditions are well known to the skilled person.

When the eukaryotic cell is yeast, this induction can be achieved by transferring the yeast into a sporulation medium, in particular from a rich medium to a sporulation medium, said sporulation medium preferably lacking a fermentable carbon or nitrogen source, and incubating the yeast in the sporulation medium for a time sufficient to induce double-strand breaks. Initiation of the meiotic cycle depends on several signals: the presence of the two sex-type alleles MATα/α nd MATα, the absence of a source of nitrogen and fermentable carbon.

As used in this document, the term ‘rich medium’ refers to a culture medium comprising a source of fermentable carbon and a source of nitrogen, as well as all the nutrients necessary for yeast to multiply by mitotic division. This medium can be easily chosen by the skilled person and can, for example, be selected from the group consisting of YPD medium (1% yeast extract, 2% bactopeptone, and 2% glucose), YPG medium (1% yeast extract, 2% bactopeptone, and 3% glycerol) and a synthetic complete (SC) medium (Treco and Lundblad, 2001, Curr. Protocol. Mol. Biol., Chapter 13, Unit 13.1).

As used herein, the term ‘sporulation medium’ refers to any medium that induces the entry into meiosis prophase of yeast cells without vegetative growth, in particular to a culture medium that does not include a fermentable carbon source or a nitrogen source but does include a source of carbon that can be metabolized by respiration such as acetate. This medium can be easily selected by the skilled person and can, for example, be selected from the group consisting of KAc 1% medium (Wu and Lichten, 1994, Science, 263, pp 515-518), SPM medium (Kassir and Simchen, 1991, Meth. Enzymol., 194, pp 94-110) and the sporulation media described in the paper by Sherman (Sherman, Meth. Enzymol., 1991, 194, pp 3-21).

According to a preferred embodiment, before being incubated in the sporulation medium, the cells are grown for a few division cycles in a pre-sporulation medium so as to obtain efficient and synchronous sporulation. The pre-sporulation medium can be easily chosen by the person skilled in the art. This medium can be, for example, the SPS medium (Wu and Lichten, 1994, Science, 263, pp 515-518).

The choice of media (rich medium, pre-sporulation medium, sporulation medium) depends on the physiological and genetic traits of the yeast strain, particularly if the strain is auxotrophic for one or more compounds.

Once the cell has entered prophase I of meiosis, the meiotic process can continue until four daughter cells with the desired recombinations are produced.

Alternatively, when the eukaryotic cell is a yeast, and in particular a yeast of the genus Saccharomyces, the cells can be returned to growth conditions to resume a mitotic process. This phenomenon, called ‘return-to-growth’ or ‘RTG’, has been previously described in patent application WO 2014/083142 and occurs when cells that entered meiosis in response to nutritional deficiency are returned to the presence of a carbon and nitrogen source after the formation of Spo11-dependent double-strand breaks but before the first division of meiosis (Honigberg and Esposito, Proc. Nat. Acad. Sci USA, 1994, 91, pp 6559-6563). Under these conditions, they interrupt their progression through the stages of meiotic differentiation to resume a mitotic growth mode while inducing the recombinations sought during the repair of double-strand breaks (Sherman and Roman, Genetics, 1963, 48, pp 255-261; Esposito and Esposito, Proc. Nat. Acad. Sci, 1974, 71, pp 3172-3176; Zenvirth et al, Genes to Cells, 1997, 2, pp 487-498).

The process may further comprise obtaining the cell(s) having the desired recombination(s). When the cell is a yeast cell, the process may further comprise a step of culturing and/or multiplying the cell or cells having the desired recombination(s). When the cell is a plant cell, the process may further comprise a somatic embryogenesis step, i.e., the regeneration of a plant embryo from a callus comprising the cells having the desired recombination(s).

The process according to the invention can be used in all applications where it is desirable to improve and control meiotic recombination phenomena. In particular, the invention makes it possible to associate, in a preferential manner, genetic traits of interest. This preferential association makes it possible, on the one hand, to reduce the time required for their selection, and on the other hand, to generate possible but unlikely natural combinations. Finally, according to the chosen embodiment, the organisms obtained by this process can be considered as non-genetically modified (non-GMO) organisms.

According to another aspect, the present invention relates to a process for generating variants of a eukaryotic organism, with the exception of humans, preferably a yeast or a plant, even more preferably a yeast, in particular a yeast strain of industrial interest, comprising
introduction into a cell of said organism of:
a) a fusion protein, a nucleic acid, an expression cassette, or a vector as described above;
and
b) one or more guide RNAs or one or more nucleic acids encoding said guide RNAs, said guide RNAs comprising a nuclease binding RNA structure associated with a CRISPR system of the fusion protein and a sequence complementary to the targeted chromosomal region; and
induction of entry into prophase I of meiosis of said cell;
obtaining cell(s) with the desired recombination(s) in the targeted chromosomal region(s);
and
genesis of a variant of the organism from said recombinant cell.

In this process, the term ‘variant’ is to be understood broadly to mean an organism with at least one genotypic or phenotypic difference from the parent organisms.

Recombinant cells can be obtained by allowing meiosis to continue until spores are obtained, or, in the case of yeast, by returning the cells to growth conditions after the induction of double-strand breaks in order to resume a mitotic process.

When the eukaryotic cell is a plant cell, a plant variant can be generated by fusion of plant gametes, at least one of the gametes being a cell recombined by the method according to the invention.

The present invention also relates to a process for identifying or locating genetic information encoding a trait of interest in a eukaryotic cell genome, preferably a yeast or plant, comprising:
introduction into the eukaryotic cell of:
a) a fusion protein, a nucleic acid, an expression cassette, or a vector as described above;
and
b) one or more guide RNAs or one or more nucleic acids encoding said guide RNAs, said guide RNAs comprising a nuclease binding RNA structure associated with a CRISPR system of the fusion protein and a sequence complementary to the targeted chromosomal region; and
induction of entry into prophase I of meiosis of said cell;
obtaining cell(s) with the desired recombination(s) in the targeted chromosomal region(s);
and
analysis of the genotypes and phenotypes of the recombinant cells in order to identify or locate the genetic information encoding the trait of interest.

Preferably, the trait of interest is a quantitative trait of interest (QTL). A quantitative trait locus (QTL) is a variably sized region of DNA that is closely associated with a quantitative trait, i.e., a chromosomal region where one or more genes responsible for the trait in question are located. These quantitative traits usually relate to a phenotypic trait. QTL analysis allows the link between a genetic variation and a phenotypic variation to be assessed.

The present invention finally relates to a kit comprising a fusion protein, a nucleic acid, an expression cassette, or an expression vector according to the invention, or a host cell transformed or transfected with a nucleic acid, an expression cassette, or an expression vector according to the invention. It also relates to the use of said kit to implement a process according to the invention, in particular to (i) induce targeted meiotic recombinations in a eukaryotic cell, (ii) generate variants of a eukaryotic organism, and/or (iii) identify or locate genetic information encoding a trait of interest in a eukaryotic cell genome.

In the peptide sequences described in this document, the amino acids are represented by their one-letter code according to the following nomenclature: C: cysteine; D: aspartic acid; E: glutamic acid; F: phenylalanine; G: glycine; H: histidine; I: isoleucine; K: lysine; L: leucine; M: methionine; N: asparagine; P: proline; Q: glutamine; R: arginine; S: serine; T: threonine; V: valine; W: tryptophan and Y: tyrosine.

In certain embodiments, all the identity percentages mentioned in this application may be fixed at at least 90%, at least 95%, at least 96%, at least 97%, at least 98% or at least 99%, identity. In particular, the embodiments in which all the percentages of sequence identity are at least 90%, at least 95%, at least 96%, at least 97%, at least 98% or at least 99%, sequence identity are considered as being described.

The following examples are presented for illustrative and non-limiting purposes and serve to illustrate the invention.

### Examples

### Materials and Methods

Construction of the P_{REC8}-NLS-FnCPF1-SPO11^{Y135F}-6×his-3×Flag-T_{ADH1} Cassette

The humanized version of the CPF1 gene of Francisella novicida carried by plasmid pY004 (pcDNA3.1-hFnCpf1) was obtained from the Addgene platform (http://n2t.net/addgene:69976; RRID:Addgene_69976) Zetsche, B., et al, 2015, Cell, 163, 759-771. Plasmid pAS604 containing the P_{ADH1}-NLS-FnCPF1-T_{ADH1} fragment was constructed by cloning the FnCPF1 sequence (PCR amplified from pY004) into plasmid pAS565 linearized with ApaI-NdeI enzymes. To fuse the C-terminal fragment of FnCpf1 with the N-terminus of the Spo11^{Y135F} protein, the SpeI-XmaI fragment of plasmid pAS532 was replaced with the SpeI-XmaI fragment of plasmid pAS604 containing the nuclear localization signal (NLS; GGMAAPKKKRKVDGG SEQ ID NO: 34) sequence and the coding portion of the FnCPF1 protein. Thus, the resulting plasmid, pAS608, contains the P_{ADH1}-NLS-FnCPF1-SPO11^{YT135F}-6×His-3×Flag-T_{ADH1} cassette. Then, the P_{ADH1} promoter was replaced by the meiosis-specific P_{REC8} promoter cloned by Gibson reaction into the SpeI-XhoI-digested pAS604 vector. Thus, the final plasmid, pAS628, contains the P_{REC8}-NLS-FnCPF1-SPO11^{Y135F}-6×His-3×Flag-T_{ADH1} cassette in which the NLS and the N-terminus of FnCpf1 are separated by a linker (GIHGVPAA, SEQ ID NO: 35). Moreover, the FnCpf1 (SEQ ID NO. 3) and Spo11^{Y135F} (SEQ ID NO. 2) domains are separated by another linker (PEFMAMEAPGIR SEQ ID NO: 36). Finally, the 6×His-3×Flag (HHHIHHGDYKDDDDKDYKDDDDKDYKDDDDK* SEQ ID NO: 37) is placed at the C-terminus of the SPO11^{T13}5F coding part.

Construction of the crRNA Expression Plasmid for Cpf1

The FnCpf1 crRNA expression cassette was designed from the plasmid pUD628 (Swiat, et al, 2017 Nucleic acids research, 45, 12585-12598); crRNA expression in it is controlled by the SNR52 promoter (P_{SNR52}) and the SUP4 terminator (T_{SUP4}). The P_{SNR52}-DR-DR-T_{SUP4} fragment in which the DRs are the 19-nucleotide direct repeats flanking the guide sequence was synthesized by GenScript. Then the SacI-XhoI fragment of the P_{RPR1}-gARN_handle-T_{RPR1} plasmid (Farzadfard, et al (2013) ACS synthetic biology, 2, pp 604-613) was replaced with the P_{SNR52}-DR-DR-T_{SUP4} sequence to create the plasmid pAS603. To obtain a doxycycline-inducible crRNA expression system, the tetO sequence (assembled with a pair of oligonucleotides) was inserted into the P_{SNR52} promoter to obtain plasmid pAS622. Based on the analysis of the architecture of P_{SNR52} (Guffanti, et al (2006). J Biol Chem, 281, pp 23945-23957) the 17-nucleotide sequence located at position-160, -176 of the transcription start site and the A-Box element of the promoter, was replaced by the 19-nucleotide tetO sequence as described previously for the RPR1 promoter (Bak, et al (2010) BMB Rep, 43, pp 110-114). Construction of a doxycycline-inducible crRNA expression system was completed by ‘Gibson’ cloning of the PCR-amplified TetR gene from plasmid pRS416gt (Smith et al (2016) Genome Biol, 17, pp 45) into the XhoI-linearized vector pAS622. The final plasmid pAS631 thus contains the doxycycline-inducible P_{SNR52}-tetO-DR-DR-T_{SUP4}-TetR crRNA expression system. The 25-nucleotide guide sequence (5′ GTCCGTGCGGAGATATCTGCGCCGT 3′ SEQ ID NO: 45) targeting the Gal4_{UAS-B,C} site in the GAL2 gene promoter was inserted between the DR sequences by Gibson reaction into the BglII-linearized plasmid pAS631.

Construction of Saccharomyces cerevisiae Yeast Strains.

Genotype of the Saccharomyces cerevisiae strains used:

AND3482: MATα/α trp1/trp1::hisG pGAL2_AB²CD²E/pGAL2_ab²cD²E tGAL2::HphMX/−pEMP46::NatMX/−his4/″leu2/″ura3/″.

AND3538: MATα/α trp1::pAS628(CPF-SPO11^{Y135F}-6×His-Flag-TRP1-KanMX)/trp1::hisG pGAL2_AB²CD²E/pGAL2_ab²cD²E tGAL2::HphMX/−pEMP46 pEMP4:NatMX/−his4/″leu2/″ura3/″.

ANT2951: MATα/α trp1::pAS628(CPF1-SPO11Y^{135F}-6×His-Flag-TRP1-KanMX)/trp1::hisG pGAL2_AB²CD²E/pGAL2_ab²cD²E tGAL2::HphMX/−pEMP46::NatMX/−his4/″leu2/″ura3/″+plasmid pAS632-crARN_{UAS-B,C}::LEU2.

AND3535: MATα/α trp1::pAS628(CPF-SPO11^{Y135F}-6×His-Flag-TRP1-KanMX)/trp1::hisG pGAL2_AB²CD²E/pGAL2_ab²cd²e tGAL2::HphMX/−pEMP46::NatMX/−his4/″leu2/″ura3/″.

ANT2945: MATα/α trp1::pAS628(CPF-SPO11^{Y13SF}-6×His-Flag-TRP1-KanMX)/trp1::hisG pGAL2_AB²CD²E/pGAL2_ab²cd²e tGAL2::HphMX/−pEMP46::NatMX/−his4/″leu2/″ura3/″+plasmid pAS632-crARN_{UAS-B,C}::LEU2.

AND2549: MATα/α trp1/trp1::hisG tGAL2::HphMX/−pEMP46::NatMX/−his4/″leu2/″ura3/″.

The introduction of P_{REC8}-FnCPF1-SPO11^{Y135F}-6×His-Flag-T_{ADH1}-TRP1-KanMX cassettes into the yeast S. cerevisiae genome was performed as described by Sarno, R et al (2017) Nucleic acids research, 45, e164. Plasmids pAS533 and pAS628 carrying P_{REC8}-FnCPF1-SPO11^{Y135F}-6×His-Flag-T_{ADH1}-TRP1-KanMX cassettes, respectively, were linearized by XbaI restriction enzyme and integrated at the TRP1 locus by cell electroporation.

The sgRNA and crRNA expression plasmids carrying the LEU2 selection marker were introduced into Saccharomyces cerevisiae AND3535, AND3538, AND3596 cells, auxotrophic for leucine (leu2⁻) by electroporation and the transformants selected on plates containing synthetic complete medium lacking leucine. Integration junctions of FnCPF1-SPO11^{Y135F} expression cassettes at the TRP1 locus were verified by PCR.

Recombination frequency around the GAL2 gene (chromosome XII) is measured between the NatMX cassette integrated at position 287 725 in the EMP46 gene promoter (pEMP46) and the HphMX cassette integrated at position 292 068 in the GAL2 gene terminator (tGAL2).

### Induction of Meiosis and Sporulation

First, diploid cells are inoculated in a rich liquid medium (YPD) or in a synthetic complete medium lacking leucine (SC-Leu) in order to maintain the plasmids carrying the LEU2 selection marker. The cells are grown under shaking at 30° C. for 24 hours (‘Mitotic’ point). Then the saturated SC-Leu liquid cultures are diluted in SPS pre-sporulation medium (2-16×10⁵ cells/ml) and cultured under shaking at 30° C. for −15 hours. Cultures that have reached an OD₆₀₀ between 2 and 4 are centrifuged, washed twice with water, and transferred to sporulation medium (KAc 1%) with a final OD₆₀₀ of 1. This is the T₀ point of meiotic progression. To induce expression of crRNAs doxycycline hyclate is added to a final concentration of 10 μg/ml in the sporulation medium at time T₀.

### Growth and Sporulation Media

YPD, SPS pre-sporulation and KAc 1% sporulation media are described in the reference Murakami et al, (2009) Methods Mol Biol, 557, pp 117-142. YPD medium consists of yeast extract (1%), peptone (2%) and glucose (2%). Synthetic complete medium without leucine is composed of yeast nitrogen base (0.17%), ammonium sulphate (0.5%), synthetic drop-out mix without leucine (0.16%) and glucose (2%).

### Southern Blot and Quantifications of DSBs and Recombinant Molecules

Genomic DNA extraction as well as detection of DSBs and recombinant molecules by Southern blot were performed as described in the references Murakami, et al, (2009) Methods Mol Biol, 557, pp 117-142, and Sarno, et al (2017) Nucleic acids research, 45, e164.

### Genetic Analysis of Tetrads

Four-spore tetrads were dissected on YPD plates after 48 hours of incubation in sporulation medium. Segregation of NatMX and HygMX markers is visualized by replicating colonies on YPD plates containing nourseothricin (100 mg/l) or hygromycin (300 mg/l), respectively.

### Results

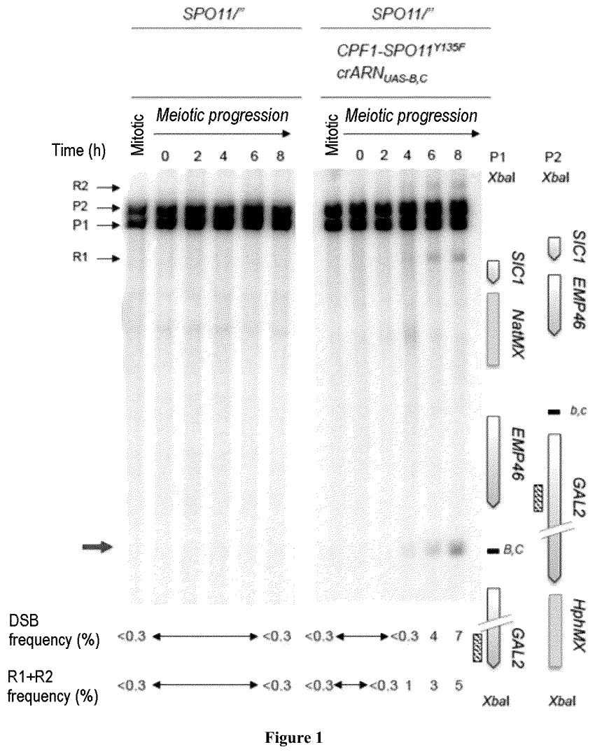

The Cpf1-Spo11^{Y135F} fusion stimulates the formation of meiotic DSBs and their repair by homologous recombination in the GAL2 gene promoter region (FIG. 1). Southern blot analysis of double-strand breaks (DSBs) and recombinant molecules in cells expressing the Cpf1-Spo11^{Y135F} fusion. Cells are harvested during mitotic growth and during meiotic progression (t=0, 2, 4, 6 and 8 hours). The gene encoding the Cpf1-Spo11^{Y135F} fusion protein is induced in diploid SPO11/SPO11 cells that contain the wild-type target sequence pGAL2B,C(5′ ACGGCGCAGATATCTCCGCACGGAC 3′, SEQ ID NO: 43) on the parental chromosome P1 and a mutated Cpf1 cut-resistant target sequence pGAL2_{b,c} on the parental chromosome P2 (5′ AttcCGCAGATATCTCCGCAtatAC 3′, SEQ ID NO: 44). To induce expression of crRNAs doxycycline hyclate is added to the meiotic culture (1% KAc) at time 0 for a final concentration of 10 μg/ml. Genomic DNA is extracted from yeast cells, digested with the restriction enzyme XbaI, deposited on an agarose gel (0.6%) and separated by electrophoresis, then transferred to a nitrocellulose membrane and hybridized with a radioactive probe located in the coding region of the GAL2 gene. The position of the DNA fragments corresponding to the parental chromosomes (P1 and P2) and to the recombinant molecules (R1 and R2) is indicated on the left side of the gel. The thick black arrow indicates the DSBs induced by Cpf1-Spo11^{Y135F} in the promoter of the GAL2 target gene at the Gal4UAS-B,c sites on chromosome P1. The map of the GAL2 region shows the open reading phases (arrows indicating the direction of transcription), the heterozygous genetic markers NatMX and HphMX located in trans of the GAL2B,C target site as well as the position of the probe (hatched rectangle). The frequency of DSBs as well as the sum of the frequencies of the recombinant molecules R1 and R2 are indicated below the gel. The minimum detection threshold for DNA molecules is 0.3%.

Strain genotype:
AND3482: MATα/α trp1 trp1::hisG pGAL2_AB²CD²EIpGAL2_ab²cd²e tGAL2:: HphMX/−pEMP46::NatMX/−his4/″leu2/″ura3/″
ANT2951: MATα/α trp1::pAS628(CPF1-SPO11^{Y135F}-6×His-Flag-TRP1-KanMX)/trp1::hisG pGAL2_AB²CD²E/pGAL2_ab²cD²E tGAL2::HphMX/−pEMP46::NatMX/−his4/″leu2/″ura3/″+plasmid pAS632-crRNA_{UAS-B,C}::LEU2

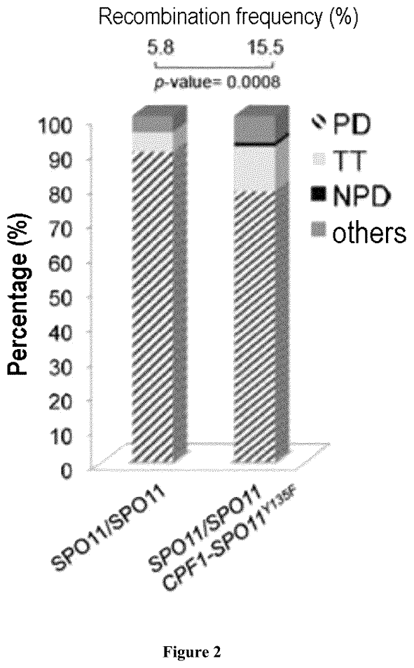

The Cpf1-Spo11^{Y135F} fusion stimulates meiotic recombination in the target region of the GAL2 gene promoter (FIG. 2). SPO11 diploid cells possess the heterozygous markers NatMX and HphMX on either side of the target regions. Their segregation was followed in the meiosis products by dissection of 4-spore tetrads. The NatMX gene confers resistance to nourseothricin (Nat^{R}) and the HphMX gene confers resistance to hygromycin (Hyg^{R}). In the absence of recombination between the NatMX and HygMX markers, the four meiosis products are of parental type: 2 Nat^{R} Hyg^{S}, 2 Nat^{S} Hyg^{R} (parental ditypes PD). On the other hand, one crossover event between markers will lead to a tetratype (TT) tetrad (1 Nat^{R} Hyg^{S}: 1 Nat^{S} Hyg^{S}, Nat^{R} Hyg^{R}, 1 Nat^{S} Hyg^{R}) while two crossover events involving the 4 chromatids in the same meiotic cell will lead to a non-parental ditype (NPD) tetrad (2 Nat^{S} Hyg^{S}, 2 Nat^{R} Hyg^{R}). Other types of segregation (called Others) have been observed. They correspond to complex events where markers segregate in a non-Mendelian manner with combinations of 4:0 and/or 0:4 segregation revealing the existence of recombinations that took place before meiosis and 3:1 and 1:3 segregations revealing the gene conversions of NatMX and/or HygMX cassettes. Respectively, 235 and 295 four-spore tetrads were dissected from WT and CPF1-SPO11^{Y135F}cells. The recombination frequency between markers is the ratio (TT+NPD)*100/(PD+TT+NPD).

Genotype of strain AND3482: MATα/α trp1/trp1::hisG pGAL2_AB²CD²EIpGAL2_ab²cd²e tGAL2::HphMX/−pEMP46::NatMX/−his4/″leu2/″ura3/″.

Genotype of strain ANT2945: MATα/α trp1:::pAS628(CPF-SPO11^{Y35F}-6×His-Flag-TRP1-KanMX)/trp1::hisG pGAL2_AB²CD²EIpGAL2_ab²cd²e tGAL2::HphMX/−pEMP46::NatMX/−his4/″leu2/″ura3/″ and contains the pAS632-crRNA_{UAS-B,C}::LEU2 plasmid.

The NatMX and HphMX cassettes were integrated into the GAL2 terminator (tGAL2) between positions 292 068 and 292 069, and into the EMP46 promoter (pEMP46) between positions 287 725 and 287 726, respectively.

1-23. (canceled)

24. A fusion protein comprising (i) a nuclease associated with a CRISPR system, and (ii) a Spo11 protein or one of the Spo11 partners involved in the formation and repair of double-strand breaks during meiosis, wherein the nuclease associated with the CRISPR system is not a Cas9 nuclease.

25. The fusion protein according to claim 24, wherein the nuclease is a nuclease associated with a class II and type V CRISPR system.

26. The fusion protein according to claim 24, wherein the nuclease associated with a CRISPR system is a Cpf1 nuclease.

27. The fusion protein according to claim 26, wherein the Cpf1 nuclease comprises a sequence selected from the sequences SEQ ID NO: 3, 4 and 22 to 33, and the variants of said sequences having at least 80% identity with one of these sequences and a Cpf1 activity.

28. The fusion protein according to claim 24, wherein the nuclease associated with a CRISPR system is deficient in nuclease activity.

29. The fusion protein according to claim 28, wherein the nuclease associated with a CRISPR system which is deficient in nuclease activity is a variant of a wild-type Cpf1 protein having at least 80% identity with said Cpf1 protein and in which the residue corresponding to the aspartate at position 832 of SEQ ID NO: 4 is substituted.

30. The fusion protein according to claim 28, wherein the nuclease associated with a CRISPR system which is deficient in nuclease activity is a variant of a sequence selected from the sequences SEQ ID NO: 3, 4 and 22 to 33, having at least 80% identity with said sequence and in which the residue corresponding to the aspartate at position 832 of SEQ ID NO: 4 is substituted by an alanine.

31. The fusion protein according to claim 24, wherein the fusion protein comprises a Spo11 protein.

32. The fusion protein according to claim 31, wherein the Spo11 protein is selected from the sequences of SEQ ID NO: 1, 10 to 21 and 40-42, and the variants thereof that comprise a sequence having at least 80% identity with one of these sequences and a Spo11 activity.

33. The fusion protein according to claim 24, wherein the fusion protein comprises a Spo11 protein deficient in nuclease activity.

34. The fusion protein according to claim 33, wherein the Spo11 protein deficient in nuclease activity is a variant of a wild-type Spo11 protein having at least 80% identity with said Spo11 protein and in which the residue corresponding to the tyrosine at position 135 of SEQ ID NO: 1 is substituted.

35. The fusion protein according to claim 33, wherein the Spo11 protein deficient in nuclease activity is a variant of one of the sequences SEQ ID NO: 1, 10 to 21 and 40-42, comprising a sequence having at least 80% identity with one of these sequences and in which the residue corresponding to the tyrosine at position 135 of SEQ ID NO: 1 is substituted by a phenylalanine.

36. The fusion protein according to claim 24, wherein the fusion protein comprises a partner of Spo11 selected from the group consisting of the proteins Rec102, MTOPOVIB/TOPOVIBL, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologs thereof, said variants having at least 80% sequence identity with one of these proteins and being capable of recruiting Spo11.

37. A nucleic acid encoding the fusion protein according to claim 24.

38. An expression cassette or vector comprising a nucleic acid according to claim 37 operably linked to a transcriptional promoter allowing expression of said nucleic acid during meiosis.

39. A host cell comprising a fusion protein according to claim 24.

40. The host cell according to claim 39, said host cell being a plant cell, a yeast cell or a fungal cell.

41. A process for inducing targeted meiotic recombinations in a eukaryotic cell, comprising:
introduction into said cell of:
a) a fusion protein according to claim 24 or a nucleic acid encoding said fusion protein, a nucleic acid encoding said fusion protein, or an expression cassette or vector comprising said nucleic acid operably linked to a transcriptional promoter allowing expression of said nucleic acid during meiosis; and
b) one or more guide RNAs or one or more nucleic acids encoding said guide RNAs, said guide RNAs comprising a nuclease binding RNA structure associated with a CRISPR system of the fusion protein and a sequence complementary to the targeted chromosome region; and
induction of entry into prophase I of meiosis of said cell.

42. A process for generating variants of a eukaryotic organism, comprising:
introduction into a cell of said organism of:
a) a fusion protein according to claim 24 or a nucleic acid encoding said fusion protein, a nucleic acid encoding said fusion protein, or an expression cassette or vector comprising said nucleic acid operably linked to a transcriptional promoter allowing expression of said nucleic acid during meiosis; and
b) one or more guide RNAs or one or more nucleic acids encoding said guide RNAs, said guide RNAs comprising a nuclease binding RNA structure associated with a CRISPR system of the fusion protein and a sequence complementary to the targeted chromosome region; and
induction of entry into prophase I of meiosis of said cell;
obtaining of cell(s) having the desired recombination(s) in the targeted chromosomal region(s); and
genesis of a variant of the organism from said recombinant cell.

43. A process for identifying or locating genetic information encoding a trait of interest in a eukaryotic cell genome, comprising:
introduction into the eukaryotic cell of:
a) a fusion protein according to claim 24, a nucleic acid encoding said fusion protein, or an expression cassette or vector comprising said nucleic acid operably linked to a transcriptional promoter allowing expression of said nucleic acid during meiosis; and
b) one or more guide RNAs or one or more nucleic acids encoding said guide RNAs, said guide RNAs comprising a nuclease binding RNA structure associated with a CRISPR system of the fusion protein and a sequence complementary to the targeted chromosome region; and
induction of entry into prophase I of meiosis of said cell;
obtaining of cell(s) having the desired recombination(s) in the targeted chromosomal region(s); and
analysis of the genotypes and phenotypes of the recombinant cells in order to identify or locate the genetic information encoding the trait of interest.

44. The process for identifying or locating genetic information encoding a trait of interest in a eukaryotic cell genome according to claim 43, wherein the trait of interest is a quantitative trait of interest (QTL).
